(19) United States
(12) Patent Application Publication
(10) Pub. No.: US 2003/0198960 A1
(43) Pub. Date: Oct. 23, 2003

(54) Signal amplifying targeted reporters for biological and chemical sensor applications

(76) Inventors: Wenhong Fan, Mountain View, CA (US); Jun Li, Sunnyvale, CA (US); Jie Han, Cupertino, CA (US)

Correspondence Address: FISH & NEAVE, 1251 AVENUE OF THE AMERICAS, 50TH FLOOR, NEW YORK, NY, 10020-1105, US

(21) Appl. No.: 10/114,776

(22) Filed: Apr. 1, 2002

(51) Int. Cl. C12Q 1/68; C08G 63/91; G01N 33/543

(52) U.S. Cl. 435/6; 525/54.2; 436/518

(57) ABSTRACT

The present invention provides targeted dendrimeric reporter molecules that provide amplified signals at electrochemical sensors, and methods of synthesis and methods of use thereof. The reporter molecules comprise a targeting moiety and at least one dendritic signal amplifier; the dendritic amplifier comprises a plurality of dendritic branches and a plurality of indicator moieties, typically pendant therefrom. The targeting moiety serves to concentrate the dendritic amplifier at a selected target, where the plural indicator provide increased signals.

### STATEMENT OF GOVERNMENT SUPPORT

This invention was made in the course of U.S. contract no. NAS2-99092 awarded by NASA. The U.S. government has certain rights in this invention.

### FIELD OF THE INVENTION

The present invention is in the field of biological and chemical sensor technology, and particularly relates to targeted dendrimeric reporter molecules that provide amplified signals at electrochemical sensors, and to methods of synthesis and methods of use thereof.

### BACKGROUND OF THE INVENTION

By directly transducing molecular recognition events into electronic signals, electrochemical sensors offer the potential of greater sensitivity, lower signal loss, higher fidelity, and better spatial resolution than do approaches that require conversion of an initial recognition signal into subsequent electrical activity. Furthermore, by obviating post-event energy conversion, electrochemical sensors should permit construction of detection devices that are smaller, mechanically less complex and more robust, than devices presently used to detect molecular recognition events.

These potential advantages, coupled with the recent explosion in the availability of nucleic acid sequence data, have motivated intensive efforts to develop electrochemical sensors that can detect and characterize nucleic acid recognition events.

For example, U.S. Pat. No. 5,312,527 discloses methods in which an electrode covalently modified with a sequence-specific single-stranded nucleic acid probe is used directly to query a sample for presence of complementary target molecules. Following exposure to sample under conditions suitable for hybridization, the electrode is placed in fluid communication with a redox active indicator having higher affinity for double-stranded than for single-stranded nucleic acids. If the queried sample had included target molecules complementary in sequence to the electrode-immobilized probe, duplex formation at the electrode acts to concentrate the redox active indicator at the electrode, increasing peak current.

Redox active indicators that have been used in this and related approaches include cationic metal complexes, nucleic acid intercalators, and agents that bind in the major or minor helical grooves, including tris(2,2′-bipyridyl)cobalt(III) perchlorate, tris(1,10-phenanthroline)cobalt(III) perchlorate, tris(2,2′-bipyridine)ruthenium(III) (Ru(bpy)₃³⁺), daunomycin, adriamycin, Hoechst 33258, intercalating metalloporphyrins, and ferrocenyl naphthalene diimide. Most of these markers do not fully discriminate between single- and double-stranded DNA, although ferrocenyl naphthalene diimide is said to have greater discriminatory power.

Ihara et al., Chem. Commun. 1997, 1609-1610, disclose the use of oligonucleotides that are rendered electrochemically active by covalent attachment to ferrocene. In a sandwich assay, the target oligonucleotide binds simultaneously to its cognate probe at the electrode surface and to a ferrocenyl oligonucleotide indicator that binds elsewhere on the target. This target-mediated dual recognition raises the electrode-local concentration of the electroactive ferrocene moiety, increasing the oxidation current. U.S. Pat. No. 6,087,100 similarly discloses covalent attachment of electron transfer moieties, such as redox active transition metal complexes, to nucleic acid probes in order to render the probes electrochemically active.

U.S. Pat. No. 5,871,918 describes several methods for electrochemical detection of nucleic acid recognition events, all of which exploit the inherent redox activity of selected naturally occurring nucleic acid bases, such as guanine, and synthetic analogues thereof.

In a first series of embodiments, the presence of double-stranded nucleic acid, indicating sequence-specific hybridization, is signaled by a slowing of the rate of oxidation of certain nucleic acid bases present within the probe, notably guanine; measurement is conducted in the presence of a redox-active agent such as ruthenium²⁺ (2,2′-bipyridine)₃ (Ru(bpy)₃²⁺). Duplex formation slows the oxidation rate by shielding the bases from the oxidizing agent, thus increasing the electron tunneling distance.

In alternative embodiments, the oxidizable base is located on the target, not the probe. The probe is preferably designed to lack oxidizable bases, and the patent discloses that redox inactive bases, such as inosine, can be substituted for guanines for this purpose. The target is designed to overhang the probe, providing a large single-stranded region available for interaction with the transition metal complex. In one embodiment, the target nucleic acids are tailed with dG using terminal transferase. Sequence-specific duplex formation thus brings the oxidizable bases into electrochemical communication with the electrode, increasing peak current.

In yet another series of embodiments, sandwich assays are used: the target is hybridized, with overhang, to the electrode-immobilized probe. A second, signaling, probe is annealed elsewhere to the target, which is only partially duplexed to the electrode-immobilized capture probe; the signal probe is dG tailed.

Kelley et al., Nucl. Acids Res. 27(24):4830-4837 (1999) and Boon et al., Nature Biotechnol. 18:1096-1100 (2000), describe electrochemical detection of DNA mismatches by direct voltammetry of redox-active intercalators noncovalently bound to DNA-modified surfaces. The electrochemical activity of the intercalators is reported by charge transport through the double-stranded DNA helix to the electrode surface; mismatches in the duplex, or other helix destabilizing events, disrupt the stacked n orbitals, reducing signal. In this approach, redox probes that intercalate into the DNA base stack are required: redox active agents that associate with DNA through purely electrostatic interactions do not yield measurable differences in the electrochemical response in the presence of base mismatches. Preferred species of intercalator include daunomycin (DM), methylene blue, and Ir(bpy) (phen) (phi)³⁺. In a further improvement, Boon et al., Nature Biotechnol. 20:282-286 (2002), use the perturbation of charge transport through the double-stranded helix to report the binding of sequence-specific DNA-binding proteins.

Park et al., Science 295:1503-1506 (2002), describe array-based electrical detection of DNA duplexes using nanoparticle probes. A short “capture” oligonucleotide of desired sequence is disposed on a silicon chip in the 20 μm gap between two fixed microelectrodes; the target oligonucleotide has contiguous recognition elements that are complementary, respectively, to the sequence of the capture oligonucleotide and to the sequence of an oligonucleotide attached to a gold nanoparticle (60 nm Au on 5 nm Ti). Duplex formation brings gold nanoparticles into the gap between microelectrodes, increasing conductivity.

These and other efforts to develop electrochemical sensors of nucleic acid recognition events are reviewed, inter alia, in Thorp, “Cutting out the middleman: DNA biosensors based on electrochemical oxidation,” TibTech 16:117-121 (1998); Wang et al., “Towards Genoelectronics: Electrochemical Biosensing of DNA Hybridization,” Chem. Eur. J. 5(6):1681-1685 (1999); Kuhr, “Electrochemical DNA analysis comes of age,” Nature Biotechnol. 18:1042-1043 (2000); Scheller et al., “Research and development in biosensors,” Curr. Opin. Biotechnol. 12:35-40 (2001); and Mascini et al., “DNA electrochemical biosensors,” Fresenius J. Anal. Chem. 369:15-22 (2001).

Because the directly transduced electrical signals generated by such electrochemical methods are typically small, variations of the above-described approaches have been described that serve to amplify the primary signal.

For example, U.S. Pat. No. 5,312,527 teaches that a linked enzymatic reaction can be used catalytically to regenerate the redox active indicator. Particularly disclosed is use of glucose oxidase to reoxidize Co(bpy)₃²⁺ to Co(bpy)₃³⁺. Regeneration of the electroactive moiety extends the duration of current flow, permitting integration over longer times to provide a larger signal.

U.S. Pat. No. 5,871,918 teaches that amplification can be effected by increasing the number of oxidizable nucleic acid bases. The patent also teaches that a redox-active mediator of appropriate potential, e.g. Ru(bpy)₃²⁺, can be used catalytically to regenerate the redox active ruthenium.

Park et al., Science 295:1503-1506 (2002), teach that amplification can be achieved by using the Au nanoparticles that are concentrated in the electrode gap to catalyze silver deposition using standard photographic chemistries.

Kelley et al., Nucl. Acids Res. 27(24):4830-4837 (1999) and Boon et al., Nature Biotechnol. 18:1096-1100 (2000), teach that signal amplification is possible by coupling direct electron transfer through the stacked bases of the duplex helix to a further redox cycle with a soluble agent, such as ferricyanide (Fe(CN)₆⁴⁻), which reoxidizes the intercalator in an electrocatalytic cycle. In such a cycle, electrons flow from the electrode surface to intercalator; the reduced intercalator in turn reduces solution-borne ferricyanide, regenerating the oxidized form of the intercalator, which becomes available for another cycle of charge transfer.

Although such schemes are capable of amplifying the primary signal in electrochemical probing of DNA, problems remain.

Many of these approaches are specific for the chosen detection scheme, constraining their use to a particular, idiosyncratic, electrochemical sensor platform.

Also, many of these amplification schemes act by increasing the duration of current flow, rather than its magnitude. Although integration over longer periods can produce a larger total signal, such cumulation commensurately increases noise. Furthermore, longer integration times increase the duration of the measurement cycle, reducing the ability of the sensor to report rapid kinetic events.

Although other described amplification schemes can increase the magnitude of current flow, these solutions, too, prove imperfect.

For example, increasing the number of oxidizable nucleobases in target nucleic acids by tailing with poly-dG, as taught in U.S. Pat. No. 5,871,918, imposes an additional sample preparation step that vitiates many of the efficiency advantages gained through direct electrochemical detection. Although sandwich assays can be designed in which the poly-dG tail is appended to a separate signaling probe, rather than to target, any such common, target-indifferent, addition of bases increases the likelihood of nonspecific cross-hybridization, leading to decreased specificity.

The signal intensity problem is further exacerbated as electrode scale decreases, since signal is often proportional to the active surface area of the electrode. Boon et al., Nature Biotechnol. 18:1096-1100 (2000); Patolsky et al., Nature Biotechnol. 19:253-257 (2001). As electrodes used in electrochemical sensors continue to shrink—and particularly as nanoscale electrodes based, e.g., on carbon nanotubes, Kong et al., Science 287:622-5 (2000), or boron-doped silicon nanowires (SiNWs), Cui et al., Science 293(5533):1289-92 (2001), are adapted for use in chemical sensors—the need for signal amplification will increase. Yet even as the need for primary signal amplification increases, the diminution in electrode surface area decreases the avenues for effecting such amplification at the electrode surface.

One solution is to increase the effective density of probes in electrochemical communication with the sensing electrode by ramifying the probes outward from the surface of the electrode, such as by use of highly-branched DNA or PNA (peptide nucleic acid) dendrimers. Wang et al., J. Am. Chem. Soc. 120:8281-8282(1998); and Wang et al., Chem. Eur. J. 5(6):1681-1685 (1999). A significant disadvantage of this approach is that distinct sequence-specific dendrimers must be synthesized for each specific target desired to be detected.

There is, therefore, a continuing need in the art for compounds, methods, compositions and kits that increase the primary signal in electrochemical detection, particularly in electrochemical detection of nucleic acid recognition events. There is a further need for signal amplifiers that can be used under aqueous conditions, and that have cross-platform applicability.

### SUMMARY OF THE INVENTION

The present invention satisfies these and other needs in the art by providing, in a first aspect, a targeted dendrimeric reporter. The targeted dendrimeric reporter comprises a targeting moiety (targeting means), and, linked thereto, a dendritic signal amplifier (dendritic signaling means).

The targeting moiety acts to concentrate the reporter in the vicinity of a molecular event desired to be detected. The dendritic signal amplifier, which comprises a plurality of dendritic branches, at least a plurality of which terminate in or otherwise include indicator moieties, serves to colocalize the appended indicators at the site of target binding. Because each of the indicator moieties so localized contributes to an aggregate signal, the dendritic ramification leads to a significant increase in the primary signal as compared to targeted, single indicator, reporters. Amplification can be as little as two-fold, and as much as 3-fold, 4-fold, 5-fold, 10-fold, and as much as 50-fold, 100-fold, even 1000-fold or more.

In typical embodiments, the dendrimeric reporter further comprises at least one linking ligand, wherein each of the dendritic amplifiers is linked to the targeting moiety by at least one of the ligands. In certain embodiments, the dendrimeric reporter further comprises a metal center, which commonly coordinates the targeting moiety and each of the linking ligands, permitting modular synthetic approaches. The metal center can, e.g., be hexacoordinating, such as Ru, or tetracoordinating, such as Pt.

The coordinating ligands can usefully possess two coordination sites (“bidentate” ligands), and are usefully selected from the group consisting of 1,2-diaminobenzenes, 1,2-diaminoethanes, bipyridines, phenathrolines, biquinoline, and pyrazines. Other coordinating ligands, to be used independently or in conjunction with the first, “bidentate”, ligands, can usefully be monodentate, and can be selected from the group consisting of pyridines, quinolines, piperidines, morpholines, pyrrolidines, pyrroles, pyrazines, thiophenes, piperazines, tetrahydrofurans, furans, phenols, and amines.

In certain embodiments particularly useful for electrochemical sensing of nucleic acid recognition events, the targeting moiety can be a nucleic acid intercalator, such as daunomycin, phenothiazinium dye, viologen, phenanthridines, phenothiazines, phenazines, acridines, anthraquinones, napththalenyls, and oxazolopyridocarbazoles.

In other embodiments, also useful for nucleic acid detection, the targeting moiety can be an oligonucleotide.

In yet other embodiments, useful for detecting proteins and other analytes, the targeting moiety can be an antibody or antigen-binding fragment thereof.

The dendritic amplifier of the targeted dendrimeric reporters of the present invention comprise a core, a plurality of dendritic branches ramifying from the core, and a plurality of indicator moieties, each of the indicator moieties bonded to a dendritic branch.

The dendritic core can usefully be selected from the group consisting of sugars and benzylamines, can in particularly useful embodiments, is a hexose sugar. The bifunctional moiety from which the dendritic branches are formed is usefully a peptide.

In embodiments useful for electrochemical sensing applications, the indicator moieties are redox active; in particularly desirable embodiments, the indicator moiety includes ferrocene or polyguanine.

In a second aspect, the invention provides methods of detecting the presence of a chosen analyte in a sample, the method comprising contacting a sample to be queried for the presence of a chosen analyte with a composition that comprises a targeted dendrimeric reporter of the present invention, and then electrochemically detecting the indicator moieties of the dendrimeric reporter molecules that are targeted to the chosen analyte.

In certain particularly useful embodiments, the analyte to be detected is a nucleic acid, and the targeting moiety is specific for double-stranded nucleic acids, such as an intercalator, for example a phenazine.

The step of electrochemically detecting analyte can comprise contacting both the sample and the intercalating dendrimeric reporter to an electrode, and detecting current flow, or altered potential, through or at the electrode. For electrochemical detection of nucleic acids, the electrode can, e.g., be covalently modified with an oligonucleotide sufficiently complementary in sequence to hybridize to the nucleic acid analyte.

The dendrimeric reporters of the present invention have cross-platform utility, and can be used in a wide variety of known electrochemical sensor applications. In each such method, the improvement comprises contacting the electrochemical sensor, in the presence of analyte, with a targeted dendrimeric reporter of the present invention.

The invention further provides, in another aspect, methods of making a targeted dendrimeric reporter. In a particularly useful approach, the method comprises linking a targeting moiety to a dendritic signal amplifier through a coordinating metal center, optionally with further intermediation by a coordinating ligand.

In a further aspect, the invention provides a kit for electrochemical detection of an analyte. The kit comprises at least one composition comprising a targeted dendrimeric reporter, and indicia that identify at least one parameter required to detect the indicator moieties of the dendrimeric reporter. Where the indicator moieties of the targeted dendrimeric reporter are redox active, the identified parameter can be an electrochemical potential of the reporter's indicators.

In embodiments particularly designed to facilitate electrochemical detection of nucleic acids, the targeted dendrimeric reporters included within the kit have binding specificity for nucleic acid.

In such embodiments, the kit can also optionally further comprise at least one composition that faciliates nucleic acid hybridization. The compositions can contain, e.g., an agent selected from the group consisting of monovalent salts, NaCl, NaOAc, divalent salts, magnesium, surface-active agents, detergents, hybridization accelerants, and cetyltrimethylammonium bromide (CTAB).

In kits designed to improve electrochemical detection of nucleic acids, the kit can optionally further comprise a composition comprising an oligonucleotide. The oligonucleotide can, e.g., be designed to facilitate sandwich recognition of an electrode-immobilized probe, or can be designed to facilitate minor differences in sequence, such as single nucleotide polymorphisms.

Whatever the targeting moiety, where the indicator of the targeted dendrimeric reporters are redox active, the kit can usefully further comprise a composition comprising a redox active agent capable of catalytically regenerating the electrochemically active indicator moiety of the targeted dendrimeric reporters.

In some embodiments, the kit can further comprise an electrochemical sensor.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIGS. 1A-1F schematize various classes of targeted dendrimeric reporters of the present invention in which a targeting moiety is linked to dendritic amplifier by intermediation of a metal center and coordinating ligand; and

FIGS. 2A-2D depict various classes of targeted dendrimeric reporters of the present invention in which one or more targeting moieties is covalently attached, either directly or indirectly, to one or more dendritic amplifiers.

### DETAILED DESCRIPTION OF THE INVENTION

Signal-amplifying Targeted Dendrimeric Reporters for Biological and Chemical Sensor Applications

In a first aspect, the present invention provides targeted dendrimeric reporters that produce amplified signals in biological and chemical sensor applications.

The targeted dendrimeric reporter comprises a targeting moiety and, linked thereto, a dendritic signal amplifier.

The targeting moiety, as more fully described below, acts to concentrate the reporter in the vicinity of a molecular event desired to be detected. The dendritic signal amplifier, which comprises a plurality of dendritic branches, at least a plurality of which terminate in indicator moieties, serves to colocalize the appended indicators at the site of target binding. Because each of the indicator moieties so localized contributes to an aggregate signal, the dendritic ramification leads to a significant increase in the primary signal as compared to targeted, single indicator, reporters. Amplification can be as little as two-fold, and as much as 3-fold, 4-fold, 5-fold, 10-fold, and as much as 50-fold, 100-fold, even 1000-fold or more. Where the targeting moiety has affinity for nucleic acids and the indicator moieties are redox active, the dendrimeric reporters of the present invention prove particularly useful in providing amplified signals for electrochemical detection of nucleic acid recognition events.

Non-covalent Molecular Topologies

Typically, the dendrimeric reporter further comprises at least one ligand that links the targeting moiety to the dendritic amplifier. The interposition of a linking ligand permits modular synthetic approaches, facilitating ready substitution of targeting moieties and/or dendritic signaling moieties. Additionally, by distancing the targeting ligand from the dendritic amplifier, the interposed linking ligand further acts to reduce potential steric hindrance of target binding.

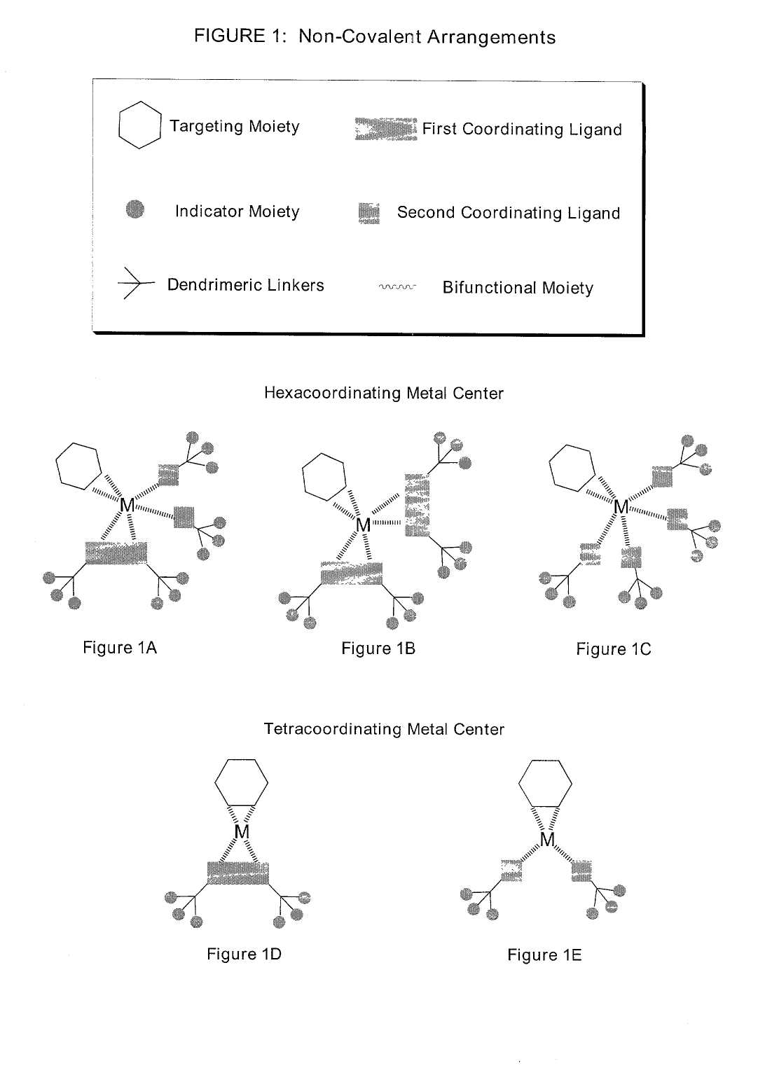

FIGS. 1A-1E schematize the general organization of several classes of targeted dendrimeric reporter embodiments of the present invention, each of which has such interposed linking ligands. In the embodiments shown, the targeted reporter further comprises a metal coordination center, “M”: the metal coordination center commonly coordinates both the targeting moiety and each of the linking ligands (which are, therefore, in these embodiments also denominated “coordinating ligands”).

In FIGS. 1A-1C, the metal center is hexacoordinating.

FIG. 1A shows a hexacoordinating metal center linking a single first coordinating ligand and two second coordinating ligands to a targeting moiety. Two dendritic amplifiers, each with trifurcating branches, are pendant from the first coordinating ligand. A single dendritic amplifier, also with trifurcating branches, depends from the second coordinating ligands. As shown, 12 indicator moieties are thus linked to the single targeting moiety.

FIG. 1B shows an alternative configuration with hexacoordinating metal center. In this organization, two second coordinating ligands are coordinated to the metal center. Each of the coordinating ligands is covalently bonded to two trifurcating dendritic amplifiers, providing a targeted reporter with 12 indicator moieties.

Yet another alternative configuration is shown in FIG. 1C, with four first coordinating ligands coordinated to the metal center, each ligand having a single trifurcating dendritic amplifier pendant therefrom.

FIGS. 1D and 1E schematize classes of embodiments formed with tetracoordinating metal centers. As shown in FIG. 1D, a single second coordinating ligand is coordinated to the metal center, with two trifurcating dendritic amplifiers pendant therefrom. In FIG. 1E, two first coordinating ligands are shown, each having a single trifurcating dendritic amplifier.

The schematic drawings of FIGS. 1A-1E are illustrative, not limiting. More generally, the targeted reporters of the present invention that have coordinating ligands are represented by either of the following formulae:

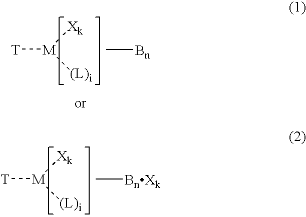

in which T is a targeting moiety, M is a metal center, X is a counterion, typically an anion, L is a coordinating ligand, and B is a dendritic amplifier.

For hexacoordinating metal centers, i is an integer from 1-5 and k is an integer from 0-4, with the specific values depending, as would be understood in the art, upon the number ligands L, the number of ions X, and the number of involved coordination sites on each of T, L and X, with the metal participating in a total of six coordinate bonds. Analogously, for a tetracoordinating metal center, i is an integer from 1-3 and k is an integer from 0-2, with the total number of coordinate bonds from the metal center four. For either hexacoordinating or tetracoordinating metal centers, n is an integer greater than or equal to 1.

When a plurality of coordinating ligands are present in a single targeted reporter, the plural coordinating ligands can be chemically distinct species, as suggested by the schemas illustrated in FIGS. 1A-1E. In preferred embodiments further described below, the targeted reporters of the present invention include up to two chemically distinct coordinating ligands.

These latter embodiments are described generally by the following formulae:

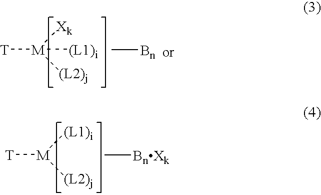

in which T is the targeting moiety, M is a metal coordination center, L1 is a first coordinating ligand, L2 is a second coordinating ligand, B is a dendritic amplifier, and X is a counterion, typically an anion.

For hexacoordinating metal centers, i is an integer from 1-5, j is an integer from 1-5, and k is an integer from 0-4, with the specific values of i, j, and k depending, as would be understood in the art, upon the number ligands L1 and L2, the number of counterions X, and the number of involved coordination sites on each of T, L1, L2, and X, with the metal participating in a total of six coordinate bonds. Analogously, for a tetracoordinating metal center, i is an integer from 1-3, j is an integer from 1-3, and k is an integer from 0-2, with the total number of coordinate bonds from the metal center equal to four. For either hexacoordinating or tetracoordinating metal centers, n is an integer greater than or equal to 1.

In preferred embodiments, the first coordinating ligand, L1, when present, has two coordination sites (and thus also referred to herein as “bidendate”), and the targeted reporter with hexacoordinating metal center can thus accommodate up to two such first coordinating ligands. In preferred embodiments, the second coordinating ligand, L2, when present, has a single coordination site (and thus referred to in the alternative herein as “monodentate”), and the targeted reporter with hexacoordinating metal center can accommodate up to five such second coordinating ligands.

### Targeting Moieties

Targeting moieties useful in the targeted dendrimeric reporters of the present invention are those that exhibit specific binding, typically noncovalent binding, to a specific binding partner.

As used herein, the term “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by more than 10- to 100-fold. When used to detect analyte, specific binding is sufficiently discriminatory when determinative of the presence of the analyte in a heterogeneous (inhomogeneous) sample. Typically, the affinity or avidity of specific binding is least about 10⁻⁷ M, with specific binding reactions of greater specificity typically having affinity or avidity of at least 10⁻⁸ M, often at least about 10⁻⁹ M, and greater. As used herein, “specific binding partners” refer to pairs of molecules, often pairs of biomolecules, that exhibit specific binding to one another.

In one series of embodiments useful for electrochemical detection of nucleic acid recognition events, the targeting moiety of the targeted reporters of the present invention bind specifically to double-stranded nucleic acids, discriminating double-stranded from single-stranded nucleic acids by at least two-fold, three-fold, 4-fold, 5-fold, or more, more typically by at least 10-fold, 20-fold, 30-fold, or even at least about 50-fold or more, and at times by at least 100-500 fold.

Such molecules include, for example, nucleic acid intercalators, such as daunomycin, phenothiazinium dye (i.e., methylene blue), viologen, phenanthridines, phenothiazines, phenazines, acridines, anthraquinones, napththalenyls, and oxazolopyridocarbazoles. Other molecules that exhibit greater affinity for double-stranded over single-stranded nucleic acids include benzo[e]pyridoindole (BePI), naphthylquinoline, imidazothioxanthone, acridines, coralyne, 4,6-diamidino-2-phenyl indole (DAPI), YOYO-1 (Molecular Probes, Eugene, OR), POPO-3 (Molecular Probes, Eugene, Oreg.), TOTO-1 (Molecular Probes, Eugene, Oreg.), ethidium bromide, PicoGreen (Molecular Probes, Eugene, Oreg.), netropsin, berenil, anthracene-9,10-diones (“anthraquinones,” i.e., daunomycin, mitoxantrone, adriamycin and their derivatives), profalvine analogues, acridines, acridinones, pyrenes, neomycin, Hoechst 33258 dye, carbocyanine dye (tetraplex-binding), and porphyrin (tetraplex-binding).

As further discussed below, targeting moieties that discriminate double-stranded from single-stranded nucleic acids find use in a wide variety of electrochemical sensing platforms.

Also useful as targeting moieties for detection of nucleic acid recognition events are nucleic acids, typically oligo- or poly-nucleotides, that include a sequence that is complementary, in the Watson-Crick duplexing sense, to a probe and/or a target nucleic acid. Nucleic acids that serve as targeting moieties can also be designed to bind nucleic acid duplexes using Hoogstein triplexing rules (see, e.g., U.S. Pat. Nos. 5,928,863 and 5,693,471).

Nucleic acids used as targeting moieties include polymers of native nucleobases in phosphodiester linkage, such as DNA or RNA, polymers that include nonnative nucleobases, such as locked nucleic acid (LNA) moieties, polymers that include nonnative linkages, and polymers that include both nonnative nucleobases and nonnative linkages.

In the present context, the term “nucleobase” covers naturally occurring nucleobases as well as non-naturally occurring nucleobases. It should be clear to the person skilled in the art that various nucleobases which previously have been considered “nonnaturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-S-methyl-4-triazolopyridin, isocytosine, isoguanin, inosine and the “non-naturally occurring” nucleobases described in U.S. Pat. No. 5,432,272. The term “nucleobase” is intended to cover each of these examples as well as analogues and tautomers thereof. Especially interesting nucleobases are adenine, guanine, thymine, cytosine, and uracil.

Among nonnative nucleobases, locked nucleic acids can find particular use in the present invention. LNAs are bicyclic and tricyclic nucleoside and nucleotide analogues and the oligonucleotides that contain such analogues. The basic structural and functional characteristics of LNAs and related analogues are disclosed, inter alia, in WO 99/14226, WO 00/56748, WO 00/66604, WO 98/39352, U.S. Pat. Nos. 6,043,060, and 6,268,490, the disclosures of which are incorporated herein by reference in their entireties.

Useful backbone modifications include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Other oligonucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage are replaced with novel groups, as in peptide nucleic acids (PNA).

In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza nitrogen atoms of the amide portion of the backbone, typically by methylene carbonyl linkages.

The uncharged nature of the PNA backbone provides PNA/DNA and PNA/RNA duplexes with a higher thermal stability than is found in DNA/DNA and DNA/RNA duplexes, resulting from the lack of charge repulsion between the PNA and DNA or RNA strand. In general, the Tm of a PNA/DNA or PNA/RNA duplex is 1° C. higher per base pair than the Tm of the corresponding DNA/DNA or DNA/RNA duplex (in 100 mM NaCl).

The neutral backbone also allows PNA to form stable DNA duplexes largely independent of salt concentration. At low ionic strength, PNA can be hybridized to a target sequence at temperatures that make DNA hybridization problematic or impossible. And unlike DNA/DNA duplex formation, PNA hybridization is possible in the absence of magnesium. Adjusting the ionic strength, therefore, is useful if competing DNA or RNA is present in the sample, or if the nucleic acid being probed contains a high level of secondary structure.

PNA also demonstrates greater specificity in binding to complementary DNA. A PNA/DNA mismatch is more destabilizing than DNA/DNA mismatch. A single mismatch in mixed a PNA/DNA 15-mer lowers the Tm by 8-20° C. (15° C. on average). In the corresponding DNA/DNA duplexes, a single mismatch lowers the Tm by 4-16° C. (11° C. on average). Because PNA probes can be significantly shorter than DNA probes, their specificity is greater.

For detecting other types of molecular events, such as presence of a protein, the targeting moiety can include other types of specific binding partners, such as biotin, any of avidin, streptavidin, neutrAvidin and captAvidin, or an antibody.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′2, and single chain Fv (scFv) fragments.

Derivatives within the scope of the term “antibody” include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies) and single-chain diabodies.

Peptides that bind specifically to a desired target, carbohydrates that exhibit specific binding to a desired target, and nonbiological organic or inorganic molecules with affinity for desired targets can also readily be used as the targeting moiety.

### Metal Centers

As noted above, a metal center can usefully be present commonly to coordinate, and thus to link, the targeting moiety and dendritic amplifier, optionally through an intervening coordination ligand.

Suitable metal centers for use in the present invention include all known and transient oxidation states of magnesium, strontium, titanium, zirconium, hafnium, vanadium, niobium, chromium, molybdenum, tungsten, manganese, rhenium, iron, ruthenium, osmium, cobalt, rhodium, iridium, nickel, palladium, platinum, copper, europium, gadolinium, terbium, dysprosium, bismuth, germanium, lanthanum, europium, zinc, cadmium, boron, aluminum, holmium, erbium, and mixtures thereof, with ruthenium or platinum presently preferred.

### Coordinating Ligands

As noted above, coordinating ligands can usefully be bidentate, such bidentate coordinating ligands usefully having the formula (5):

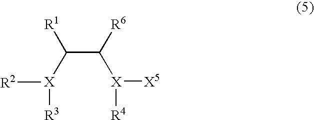

wherein X is selected from the group of heteroatoms consisting of nitrogen, oxygen, sulfur, phosphorus and selenium; R¹, R², R³, R⁴, R⁵ or R⁶ are each independently selected from the group consisting of a lone pair of electrons, hydrogen, oxygen methyl, linear alkyls, branched alkyls, cyclic alkyls, aminoalkyls, oxoalkyls, sulfoalkyls, phosphoroalkyls and selenoalkyls, any of the alkyls optionally interrupted or substituted by any of nitrogen, oxygen, sulfur, phosphorus, selenium, silicon, double and triple bonds, carbonyl, sulfonyl, sulfinyl, phosphoryl, thiocarbonyl moieties, and combination thereof.

In addition, R¹, R², R³, R⁴, R⁵ or R⁶ may together bridge with one or more of R¹, R², R³, R⁴, R⁵ or R⁶ groups to form at least one cyclized moiety within the ligand.

Examples of suitable bidentate ligands include, but are not limited to, 1,2-diaminobenzenes, 1,2-diaminoethanes, 2,2′-biquinolines, 2,3-bis(2-pyridyl)pyrazines, 2-(phenylazo)pyridines, 4,4′-dimethyl-2,2′-bipyridine, 4,4′-dichloro-2,2′-bipyridine, 2,2′-bipyridine, 4,4′-dicarboxy-2,2′-bipyridine, 4,4′-bis(dimethylamino)-2,2′-bipyridine, cycloocta[2,1-b:3,4-b′]dipyridine, 4,4′-diamino-2,2′-bipyridine, 5,5′-diamino-2,2′-bipyridine, 6,6′-diamino-2,2′-bipyridine, 4,4′-diethylenediamine-2,2′-bipyridine, 5,5′-diethylenediamine-2,2′-bipyridine, 6,6′-diethylenediamine-2,2′-bipyridine, 4,4′-dihydroxyl-2,2′-bipyridine, 5,5′-dihydroxyl-2,2′-bipyridine, 6,6′-dihydroxyl-2,2′-bipyridine, 3,4,7,8-tetramethyl-1,10-phenathroline, 4,7-diphenyl-1,10-phenanthroline, 1,7-phenanthroline, 5,6-dimethyl-1,10-phenanthroline, 4,7-dimethyl-1,10-phenanthroline, 5-bromo-1,10-phenanthroline, 5-nitro-1,10-phenanthroline, 5-chloro-1,10-phenanthroline, 2,9-dimethyl-4,7-diphenyl-1,10-phenanthroline, 2,9-dimethyl-1,10-phenanthroline, 1,10-phenanthroline, 2,9-bis(hydroxymethyl)-1,10-phenanthroline, 4,7-diamino-1,10-phenanthroline, 3,8-diamino-1,10-phenanthroline, 4,7-diethylenediamine-1,10-phenanthroline, 3,8-diethylenediamine-1,10-phenanthroline, 4,7-dihydroxyl-1,10-phenanthroline, 3,8-dihydroxyl-1,10-phenanthroline, 4,7-dinitro-1,10-phenanthroline, 3,8-dinitro-1,10-phenanthroline, 4,7-diphenyl-1,10-phenanthroline, 3,8-diphenyl-1,10-phenanthroline, 4,7-disperamine-1,10-phenanthroline, and 3,8-disperamine-1,10-phenanthroline.

Presently preferred as bidentate coordinating ligands are those having a bipyridyl backbone as shown in structure (6).

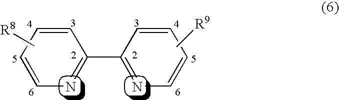

As shown, the functionalized bipyridine ligand has two nitrogen atoms for coordinating to the metal center, with R⁸ and R⁹ denoting points of functionalization and adaptation for the attachment of dendritic signal amplifiers or direct attachment of indicator moieties. R⁸ and R⁹ in structure (6) can reside on positions 3, 4, 5 and 6 either concurrently, to afford a symmetrical ligand, or non-currently to afford an asymmetrical first-coordinating ligand.

Monodentate coordinating ligands that are usefully included within the targeted reporters of the present invention can usefully have formula (7):

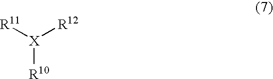

wherein X is selected from a group consisting of nitrogen, oxygen, sulfur, phosphorus and selenium; and R¹⁰, R¹¹ or R¹² are each independently selected from the group consisting of a lone pair of electrons, hydrogen, oxygen, methyl, linear alkyls, branched alkyls, and cyclic alkyls, any of the alkyls interrupted or substituted by any of nitrogen, oxygen, sulfur, phosphorus, selenium, silicon, double and triple bonds, carbonyl, sulfonyl, sulfinyl, phosphoryl, thiocarbonyl moieties, and combinations thereof. In addition, R¹⁰, R¹¹ or R¹² may together bridge with one or more of R¹⁰, R¹¹ or R¹² groups to form at least one cyclized moiety within the ligand.

Examples of suitable monodentate ligand include, but are not limited to, phenazines, pyrazines, quinolines, pyridines, piperidines, morpholines, pyrrolidines, pyrroles, thiophenes, piperazines, tetrahydrofurans, furans, phenols, imidazoles, imidazolidine, pyrimidines, anilines, triazoles, triazines, derivatives of melamines, linear, branched and cyclic alkylthiols, alkylphosphines, alkylamines, mono- and polycyclics, mono- and polycyclic aromatics, and non-natural and natural sugars, monosaccharides, carbohydrates, amino acids, peptides, and nucleobases.

Presently preferred monodentate ligands have a pyridyl structure as shown in (8):

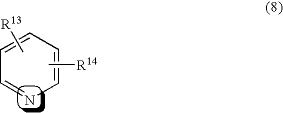

in which R¹³ and R¹⁴ can be electrophilic or nucleophilic, and adapted covalently to bond to dendritic signal amplifiers.

Examples of preferred pyridyl ligands include aminopyridines, dimethylpyridines, acetylpyridines, nitropyridines, pyridinemethanol, pyridinecarboxylic acid, pyridinedicarboxylic acid, pyridinecarboxaldehyde, and pyridinedicarboxaldehyde.

Coordinating ligands are not limited to those that are bidentate or monodentate, but can also, e.g., be tridentate, such as 4′-chloro-2,2′:6′,2″-terpyridine, 4,4′,4″-triamino-2,2′,2″-terpyridine, 4,4′,4″-triethylenediamine-2,2′,2″-terpyridine, 4,4′,4″-trihydroxy-2,2′,2″-terpyridine, 4,4′,4″-trinitro-2,2′,2″-terpyridine, and 4,4′,4″-triphenyl-2,2′,2″-terpyridine.

### Bonding of Coordinating Ligands to Dendritic Amplifiers

The coordinating ligands can be bonded to one or more dendritic amplifier moieties directly, or can be bonded indirectly through a bifunctional moiety. Whether direct or indirect bonding, the link between a linking ligand and dendritic amplifier typically involves at least one covalent bond.

Linkages between coordinating ligands and dendritic amplifiers can be facilitated by acids, bases, metal catalysis and carbodiimides. Examples of catalyzing acids include para-toluenesulfonic acid, oxalic acid, succinic acid, hydrochloric acid, sulfuric acid, nitric acid, carbonic acid as well as lewis acids and metal catalysts. Examples of bases include triethylamine, pyridine, diisopropylamine, ammonia, sodium hydroxide and hydrogen carbonate. Examples of preferred carbodiimides include DCC, CDI and EDAC.

Carbodiimides represent an efficient means of coupling amines, hydroxyls or thiols to carboxylic acid moieties under mild conditions. Scheme (1) illustrates an example wherein a first-coordinating ligand, having an electrophilic —CO₂H moiety, as in 2,2′-bipyridyl-4,4′-dicarboxylic acid, is linked to a nucleophilic dendritic amplifier moiety through an amide bond using dicyclohexylcarbodiimide (DCC):

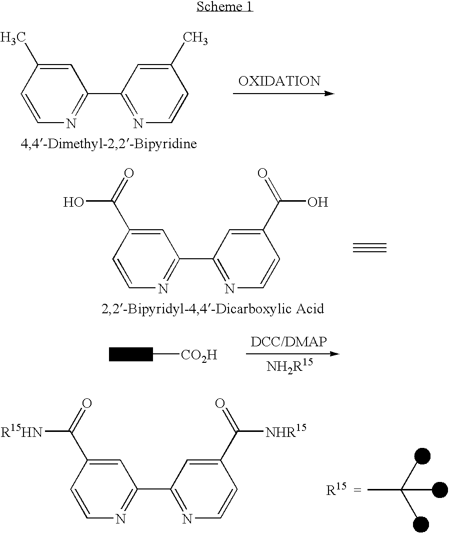

wherein R¹⁵ is a dendritic signal amplifier.

Although 2,2′-bipyridyl-4,4′-dicarboxylic acid (Sigma-Aldrich Chemical Co., Milwaukee, Wis.) is commercially available, 2,2′-bipyridyl-4,4′-dicarboxylic acid can also be obtained by oxidation of 4,4′-dimethyl-2,2′-bipyridine, as shown.

Not shown, all reactions involving carbodiimide liberate a molecule of water for each amide, ester or thioamide linkage that is formed.

Other preferred types of coupling reagent include phosphonium based reagents such as BOP, and uronium based reagents such as HBTU, TBTU, HATU, HBPyU, HBPipU PyBroP, TDBTU, TPTU, HPPyU, TOTU, BroP, PyCloP, PyCIU, PipCIU. In addition to phosphonium and uronium coupling reagents, formamidinium and imidazolidinium based coupling reagents can also be used (all available through Aldrich Chemicals, Milwaukee, Wis., USA).

Linkages can be also made without a coupling reagent in cases in which the reactivity of the electrophile has been enhanced through a chemical modification, but nonetheless synthetically equivalent.

For example, the acid chloride and N-hydroxysuccinimide ester derivatives are synthetically equivalent to the parent carboxylic acid (2,2′-bipyridyl-4,4′-dicarboxylic acid), but in a masked form, and can be used instead to achieve the same results:

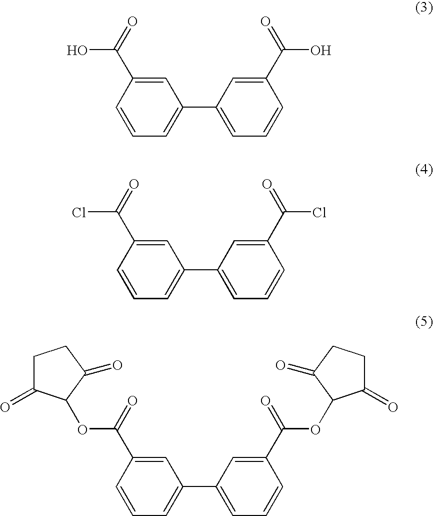

Other notable synthetic equivalents of carboxylic acids are ketenes, imidazolyl esters and mixed anhydrides.

The synthetic equivalents shown above are not intended to be limiting, nor are they intended to be biased in preference nor intend that the first-coordinating ligands must have or be synthetically equivalent to a carboxylic functionality in order to facilitate bond formation with a nucleophilic dendritic signal amplifier.

For example, by virtue of its hydroxyl groups, bishydroxyalkylbipyridine shows nucleophilic behavior, and can therefore react with an electrophilic component of a dendritic signal amplifier such as an isocyanate group, —N═C═O (Scheme 2):

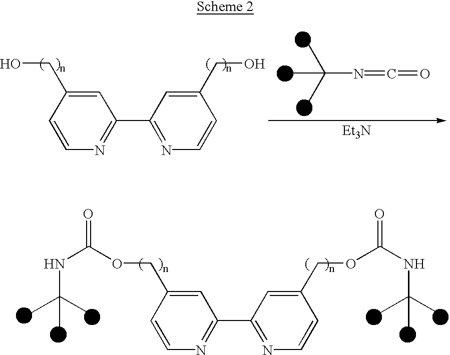

However, by reacting the hydroxyl groups with a sulfonyl group selected from a group consisting of methanesulfonyl chloride, toluenesulfonyl chloride, trifluoromethanesulfonyl chloride, and nitrobenzenesulfonyl chloride, the carbon immediately adjacent to the hydroxy group is made more electropositive or electrophilic. Scheme 3 shows a typical mesylation reaction in the presence of triethylamine (Et₃N) base:

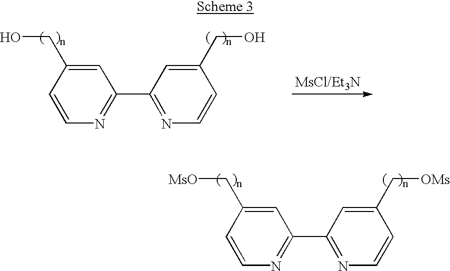

In the presence of a nucleophile, for instance, a dendritic signal amplifier, a covalent bond is created via a displacement reaction (Scheme 4):

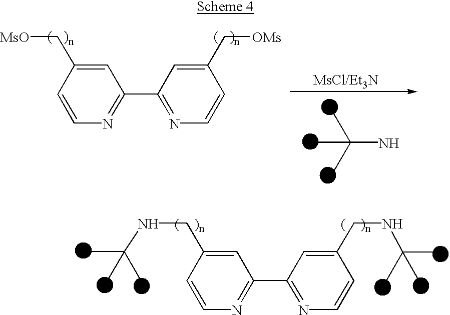

Alternatively, a coordinating ligand and dendritic signal amplifier can be linked through a bifunctional moiety. The bifunctional moieties have at least two bondable substituents that facilitate chemical attachment. Scheme (5) illustrates this alternative embodiment wherein the bifunctional moiety is a heterobifunctional moiety:

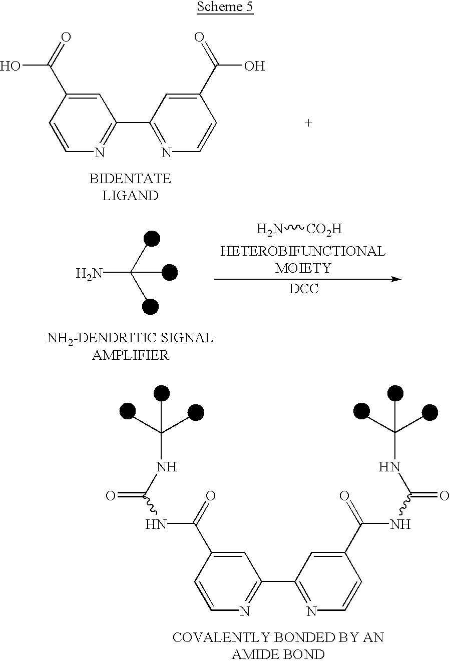

The bifunctional moiety can be selected from the group consisting of natural and non-natural amino acid, peptides and sugars as well as commercially available homobifunctional and heterobifunctional moieties.

Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA). Common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available from Pierce Chemical Co., Rockford, Ill., USA).

### Dendritic Amplifier Moieties, and Synthesis thereof

Dendritic signal amplifiers used in the targeted reporters of the present invention comprise a plurality of dendritic branches, at least a plurality of which terminate in or otherwise contain a signaling moiety.

Dendritic signal amplifiers have been described for use in other methods.

For example, U.S. Pat. No. 5,567,411 describes dendritic amplifier molecules having multiple terminal active groups stemming from a benzyl core group, such as 3,5-bis(aminomethyl)benzene and aminomethyl benzene. The dendritic amplifiers are used to amplify in vivo magnetic resonance contrast signals.

Balzani et al., Chem. Commun. 2000, 853-854, describe poly(propylene amine) dendrimers in which coordination of a single metal ion quenches the excited state of 32 terminal dansyl units.

Casado et al., Angew. Chem. Int. Ed. Engl. 39(12):2135-2138 (2000), describe poly(propylene imine) dendrimers functionalized with ferrocene or cobaltocenium moieties that are redox active.

The dendritic signal amplifiers that have previously been described, however, tend to be insoluble in aqueous solution, reducing their usefulness in biological and chemical sensor applications—particularly those that are designed to detect and characterize biologically-relevant events, such as nucleic acid hybridization—that are performed under aqueous conditions.

Furthermore, the dendritic amplifiers that have previously been described are untargeted, and must thus be used in quantities sufficient to achieve the desired effect after equilibration throughout the entirety of the bulk solution phase.

The dendritic amplifiers of the targeted reporters of the present invention, however, are preferably soluble in aqueous solution, and by tethering to a targeting moiety, are concentrated at the site of molecular recognition, permitting lower concentrations to be used.

In general, dendrimers are synthesized by the attachment of branching molecules, known as bifunctional moieties, to a core unit. Iterative polymerization affords an interior generation of layers composed of bifunctional moieties which terminate with bondable substituents at the outermost, exterior surface. For dendritic amplifiers, the bondable substituents at the exterior surface can be selected to bond either covalently or non-covalently to indicator moieties.

Suitable bifurcating, trifurcating and tetrafurcating dendritic cores for the purposes of the present invention are illustrated below:

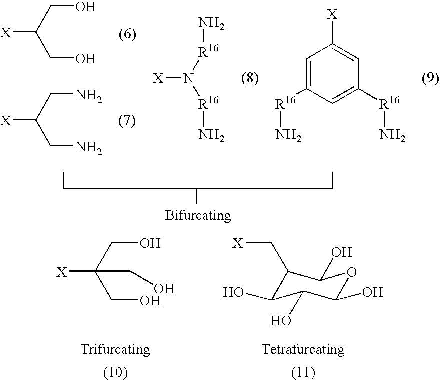

wherein X is a bondable substituent selected from the group consisting of —CO₂H, —COCl, —CO₂N(COCH₂)₂, —C═N═O, —C═N═S, —CO₂CH₃, —SH, —OH, and —NH; R¹⁶ is —CH₂—, —CO—, or —COCH₂—; and X′ is —OH, —NH₂, hydrogen or a lone pair of electrons.

The core structure dictates the degree of initial branching. Subsequently, the selection and number of bifunctional moieties, the number of polymerization cycles (or “generations”), the presence or absence of additional core molecules, which function as additional branch points, ultimately govern the size, shape, size of voids, degree of branching, solubility, overall topology, and other physical properties of the dendrimer.

Benzylamines are useful as dendritic cores for the targeted reporters of the present invention. The abondable substituent X provides a means for linking the core structure to a targeting moiety, while the bifurcating amines allow for the growth of dendrimers therefrom using bifunctional moieties:

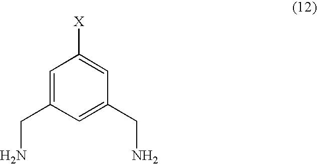

Bifunctional moieties can be attached to the amine bondable substituents using a variety of chemical bonding reactions.

Examples of some useful reactions include, but are not limited to, amidation with bifunctional moieties having a carboxyl acid bondable substituent, acylation with bifunctional moieties having an acyl halide bondable substituent, Mitsunobu reaction with bifunctional moieties having an alcoholic bondable substituent, carbamoylation with bifunctional moieties having an isocyanate bondable substituent, thiocarbamoylation with bifunctional moieties having an isothiocyanates bondable substituent.

Preferably, bifunctional moieties are attached to the benzylamine core via a Michael Addition reaction, wherein vinyl cyanide, a bifunctional moiety, adds to the free amine groups (Scheme 6). Reduction of the tetracyanobenzylamine provides a benzylamine-based dendrimer having four free amine groups from which the next generation of dendrimer can be propagated again with vinyl cyanide, or terminated in a parallel fashion by indicator (“sensing”) moieties:

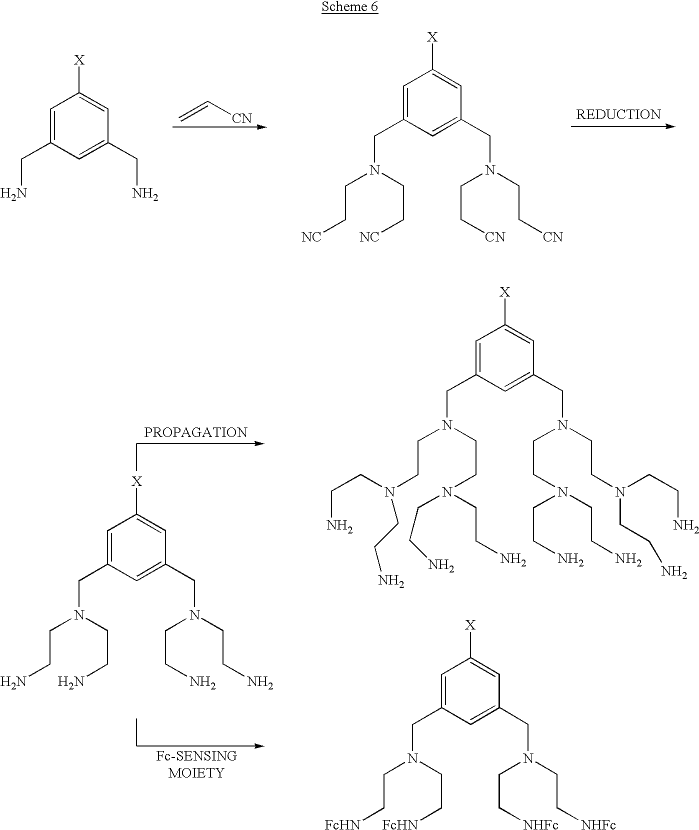

wherein X is —CO₂H, —COCl, —CO₂N(COCH₂)₂, —C═N═O, —C═N═S, —CO₂CH₃, —SH, —OH, or —NH₂; and Fc is selected from the group consisting of —COCH₂C₅H₄FeC₅H₅ and —COC₅H₄FeC₅H₅.

If, however, indicator moieties themselves, such as Fc′, wherein Fc′ is (HO₂C(CH₂)ₙC₅H₄)₂Fe and n is 0-1, act as bifunctional moieties or junctions that further join bifunctional moieties, such as NH₂CH₂CH₂NH₂, indicator moieties can be arranged in a serial fashion interrupting the contiguous organization of the dendrimeric branches, as shown in Scheme 7:

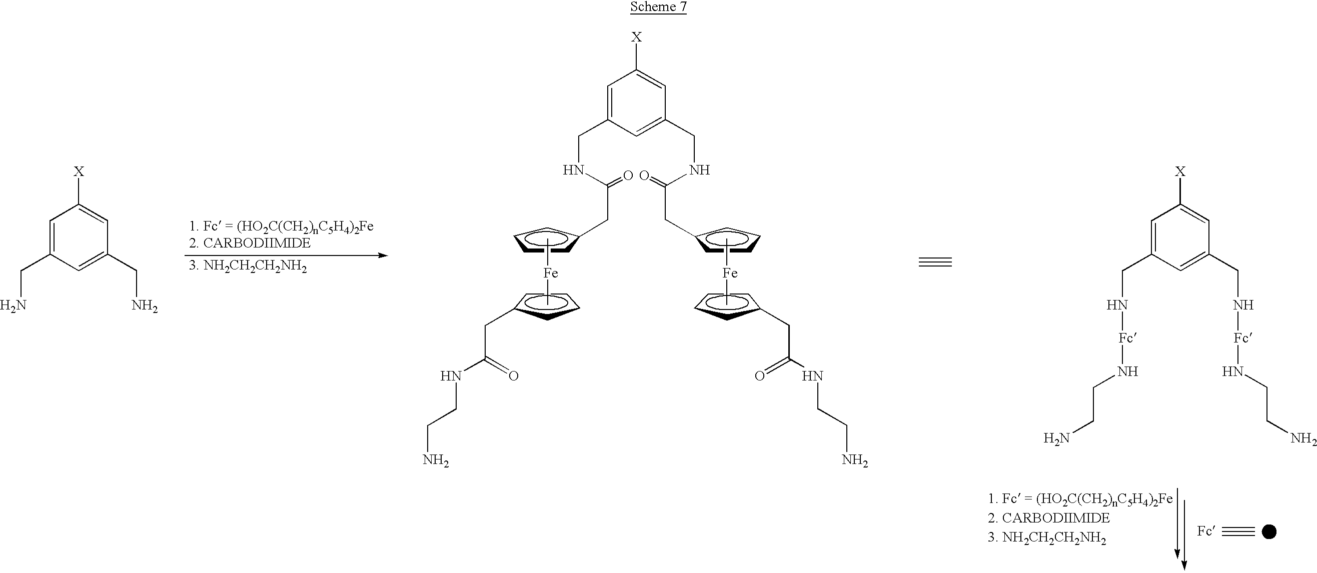

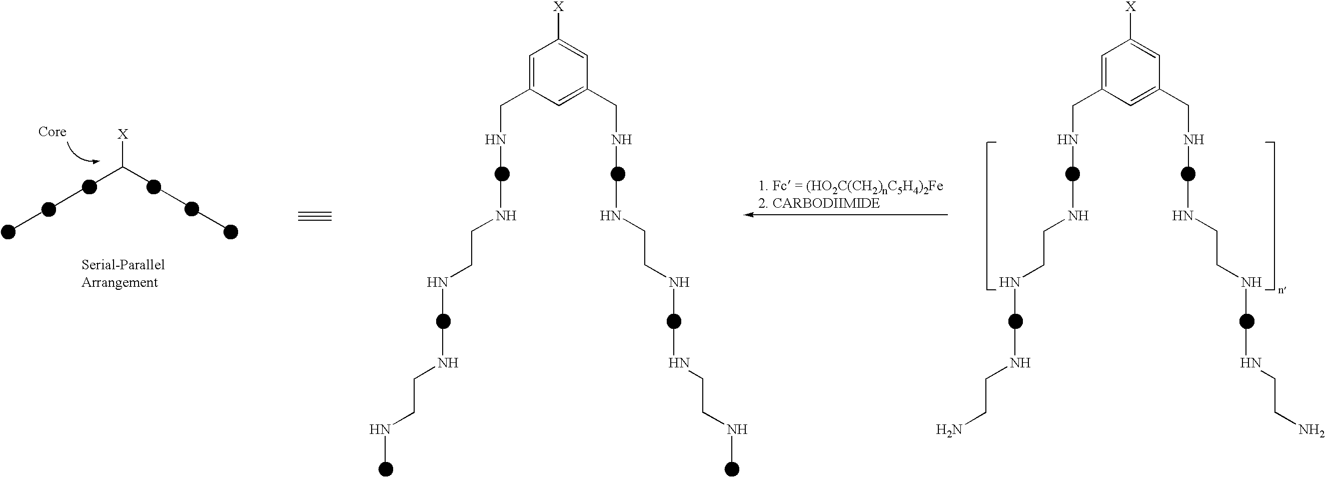

When more than one branch is interrupted with (Fc′—NHCH₂CH₂NH)_{n′} moieties, wherein n′ is 0-25, a serial-parallel arrangement of indicator moieties allows for a greater magnitude and efficiency of amplification.

In the present invention, carbohydrates, a representative example of which is shown below, prove remarkably useful as water-soluble tetrafurcating dendritic cores by virtue of their four chemically-available hydroxyls:

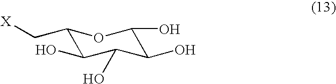

The sugar-based core has four branching points to which bifunctional moieties can be attached.

Although shown as a hexose sugar, the dendritic amplifier can also be nucleated from ribose, tetrose, and triose sugars although, as would be understood in the art, such sugars permit fewer initial branches.

In the present invention, bifunctional moieties are usefully chosen to confer aqueous solubility on the targeted reporter, since recognition events desired to be detected will often be measured in an aqueous environment under physiological conditions.

Thus, bifunctional moieties of the targeted reporters of the present invention can usefully include, but are not limited to, peptides, carbohydrates, DNA duplexes, and combinations thereof. In particular, peptides and carbohydrates, not typically found in dendritic amplifiers, are ideal bifunctional moieties for the targeted reporters of the present invention: soluble in water, they help to counteract the water insolubility of certain desirable indicator moieties, such as ferrocene.

Peptides and carbohydrates provide a remarkably flexible platform for dendrimer growth, permitting indicator moieties to be appended intermittently onto, inserted interruptingly into, or attached pendantly to each branch of the dendrimer. Scheme (8) illustrates this aspect of the invention:

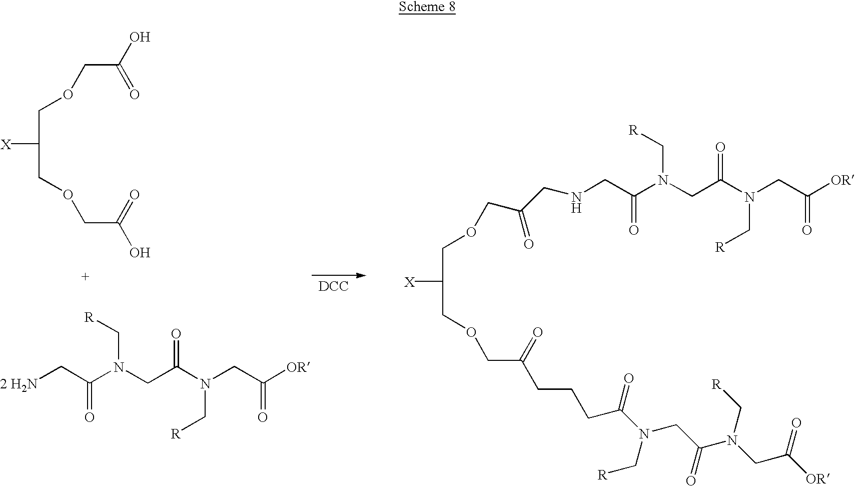

wherein R is an indicator moiety; R′ can be either an indicator moiety or a cleavable or hydrolyzable ester; and X is bondable substituent such as —NH₂, —OH or —SH.

Although dicyclohexylcarbodiimide is shown as the reagent used to couple the bifunctional peptide linkers to the bifurcating core, other coupling reagents, such as CDI and EDAC, can also be used.

Coupling reagents useful for coupling bifunctional peptide linkers to the dendritic core include phosphonium-based reagents, such as BOP, and uronium based reagents such as HBTU, TBTU, HATU, HBPyU, HBPipU PyBroP, TDBTU, TPTU, HPPyU, TOTU, BroP, PyCloP, PyCIU, PipCIU. In addition to phosphonium and uronium coupling reagents, formamidinium and imidazolidinium based coupling reagents can also be used (all available through Aldrich Chemicals, Milwaukee, Wis., USA).

In this particular example, a —CH₂CO₂H group is attached to each hydroxyl group of a pyranose sugar core to give a pyranose tetraacid molecule:

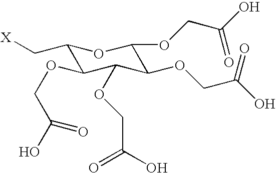

Optionally, other bifunctional moieties may also be used instead of —CH₂CO₂H groups. Other useful bifunctional moieties include homobifunctional and heterobifunctional moieties. Common homobifunctional reagents include, e.g., APG, AEDP, BASED, BMB, BMDB, BMH, BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); and common heterobifunctional cross-linkers include ABH, AMAS, ANB-NOS, APDP, ASBA, BMPA, BMPH, BMPS, EDC, EMCA, EMCH, EMCS, KMUA, KMUH, GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

However, continued polymerization of the pyranose tetraacid convergently with NH₂CH₂CH₂N(CHCO₂R′″)₂ affords dendritic signal amplifier moiety shown below, wherein R′″ is an indicator moiety or tert-butyl group:

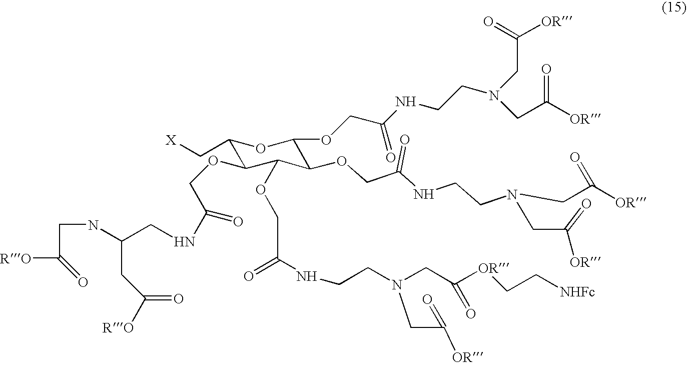

Since tert-butyl groups are readily hydrolyzable by acid, hydrolysis of the tert-butyl groups followed by successive chemical coupling with 1,4-diaminoethane and an indicator moiety, such as ferrocene (Fc), affords the dendrimer shown below:

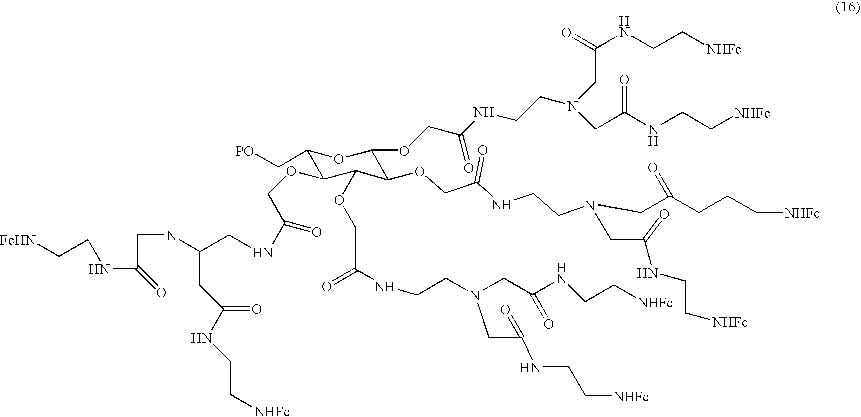

Although each iterative addition of a bifunctional moiety shown above occurs through an amide bond, other bonding modes selected from the group consisting of carbamate, urea, thioamide, thiourea, ester, and mixtures thereof, can be used to generate the dendrimeric architecture.

### Indicator Moieties

Indicator moieties (equivalently termed “signaling moieties” or “sensing moieties”) may usefully be selected from the group consisting of electroactive moieties, moieties that fluoresce, moieties that luminesce, and moieties that phosphoresce.

Suitable electroactive indicator moieties include dyes, pyrenes, fluorophores, chromophoric molecules and polyguanine. Other suitable electroactive molecules useful as indicator moieties include dicyclopentadienyl derivatives having bondable substituents, or dicyclopentadienyl metals, such as dicyclopentadienyl iron, ruthenium, osmium, bismuth, germanium, cobalt, rhodium, iridium, nickel, palladium, platinum, copper, lanthanum, europium, erbium, terbium, titanium, chromium, molybdenum, tungsten, zinc, cadmium, manganese, boron, aluminum, rhenium or mixtures thereof.

In an alternative, the bondable substituent of the bifunctional molecule can itself carry a charge, so that the entire dendrimer behaves as a polyelectrolyte. The polyelectrolyte, being charged, increases the solubility of the dendritic signal amplifier molecule in water, and permits the measurement of recognition events involving water soluble targets and analytes, such as nucleic acids, carbohydrates and proteins, even in the presence of a plurality of insoluble sensing moieties such as ferrocene.

As noted above, depending upon the choice of bifunctional moiety, the indicator moieties can be pendant from the ultimate termini of the dendritic branches in a parallel format or in a serial format. Optionally, the signaling moieties can also be arranged in a serial-parallel format:

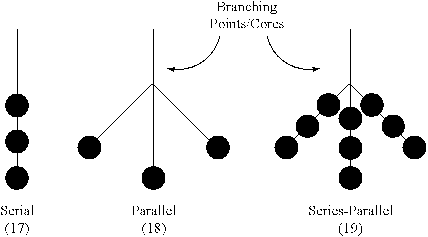

Alternative Topologies

The targeted reporters of the present invention can include a variety of alternative molecular topologies by combining those shown above. For example, it has been disclosed hereinabove that a linear polymer, having “n” repeating bifunctional moieties (Scheme 9), wherein n is

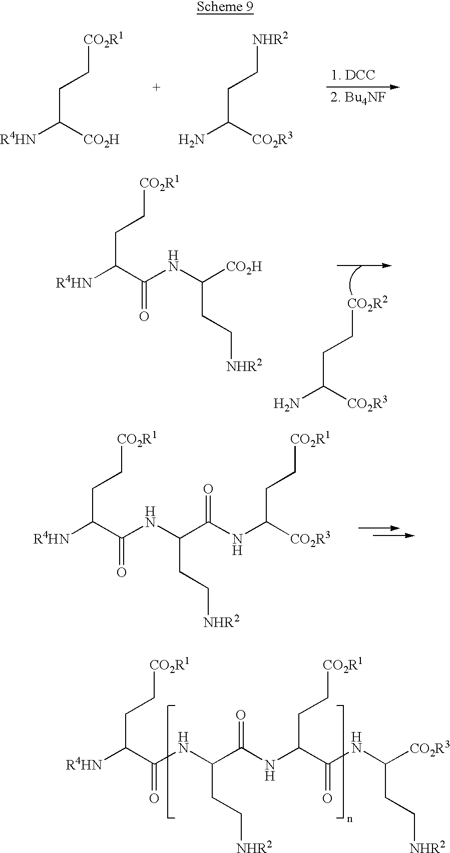

a whole number, can also have n′ number of indicator moieties serially pendant from the linear polymer by virtue of chemical appendages that function equivalently to cores (Scheme 10):

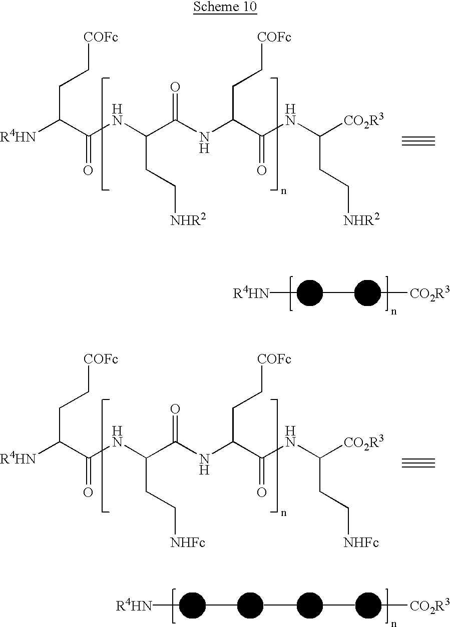

Therefore, by virtue of these —CO₂R¹, —NHR², —CO₂R³, and —NHR⁴ appendages, wherein each of R¹, R², R³ and R⁴ is independently selected from the group consisting of hydrogen, inorganic or organic cation, methyl, ethyl, benzyl, diphenylmethyl, tert-butyl, trialkylmethylsilyl, tert-butyldimethylsilyl, triphenylsilyl, 9-fluorenylmethyl, carbobenzyloxy, and tert-butoxycarbonyl, a plurality of indicator moieties Fc, wherein Fc is selected from the group consisting of —NHCH₂(C₅H₄)Fe(C₅H₅), —NHCH₂CH₂ (C₅H₄)Fe(C₅H₅), —C(O)CCH₂(C₅H₄)Fe(C₅H₅), and —C(O)(C₅H₄)Fe(C₅H₅), as well as dendritic amplifiers, having either a serial, parallel or serial-parallel format, can be appended therefrom to afford many new and novel arrangements of indicator moieties.

For example, a linear polymer, having a serial format, can be combined with other linear polymers providing an array of indicator moieties (Scheme 11):

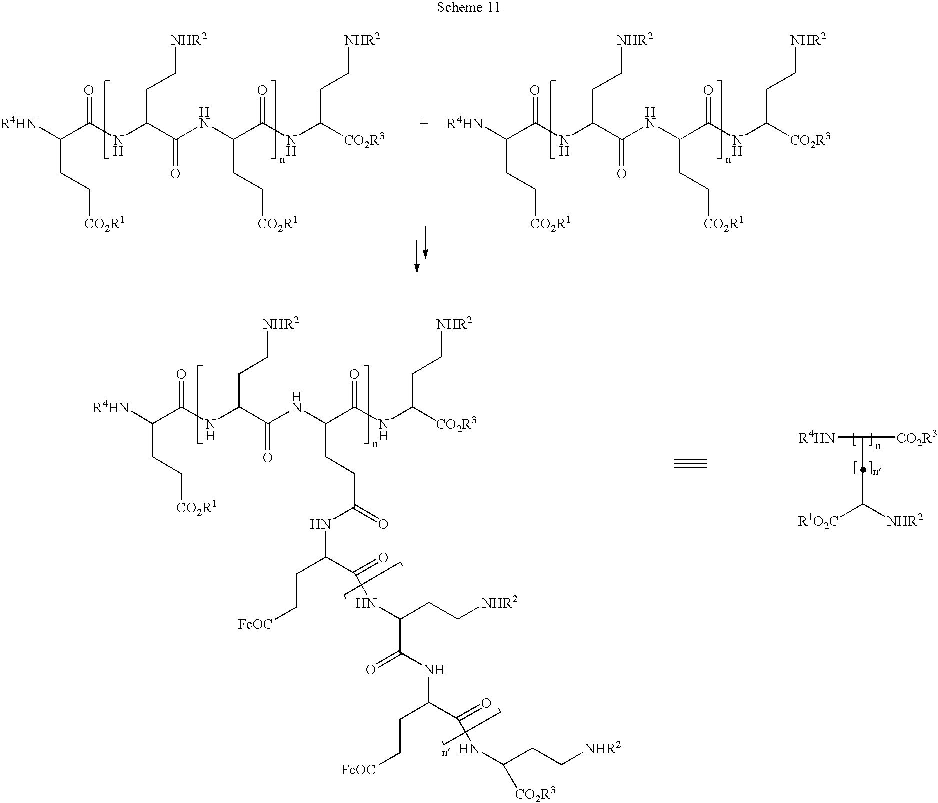

In addition, a plurality of dendritic amplifiers, arranged in a parallel format, can emanate from a main linear polymeric scaffold as shown in scheme 12:

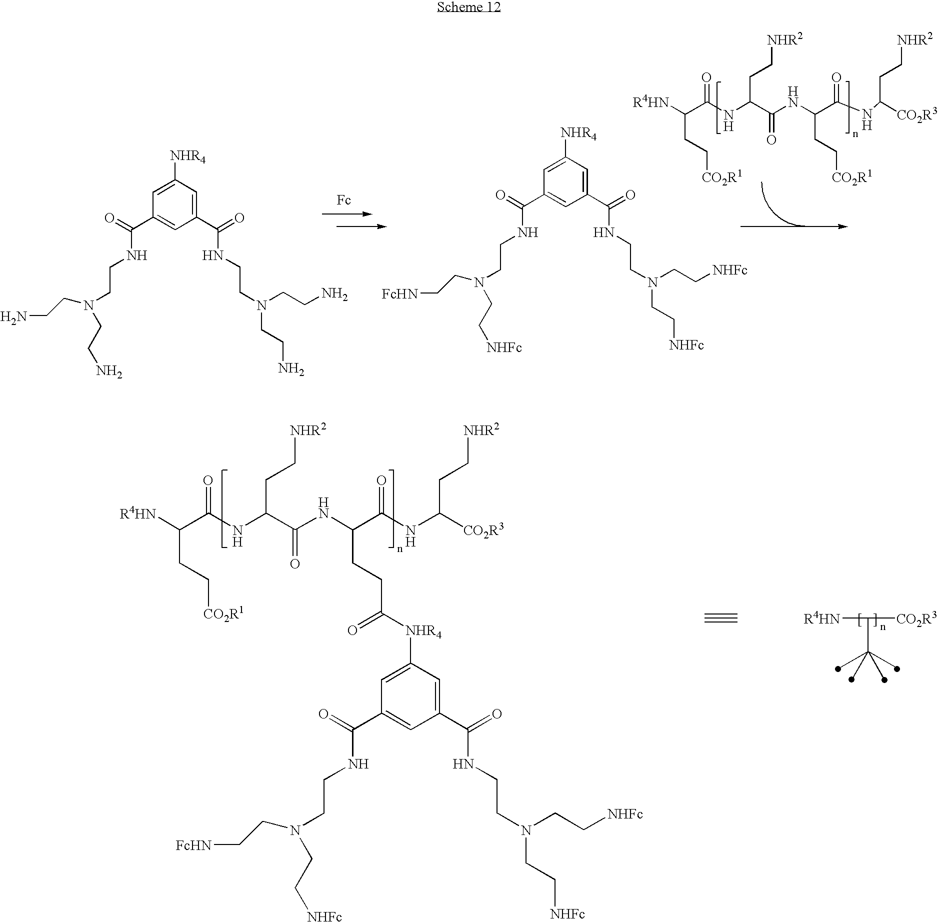

Optionally, it is within the spirit of invention to have dendritic amplifiers, arranged in a serial-parallel format, pendant from a singular polymeric scaffold as shown in scheme 13:

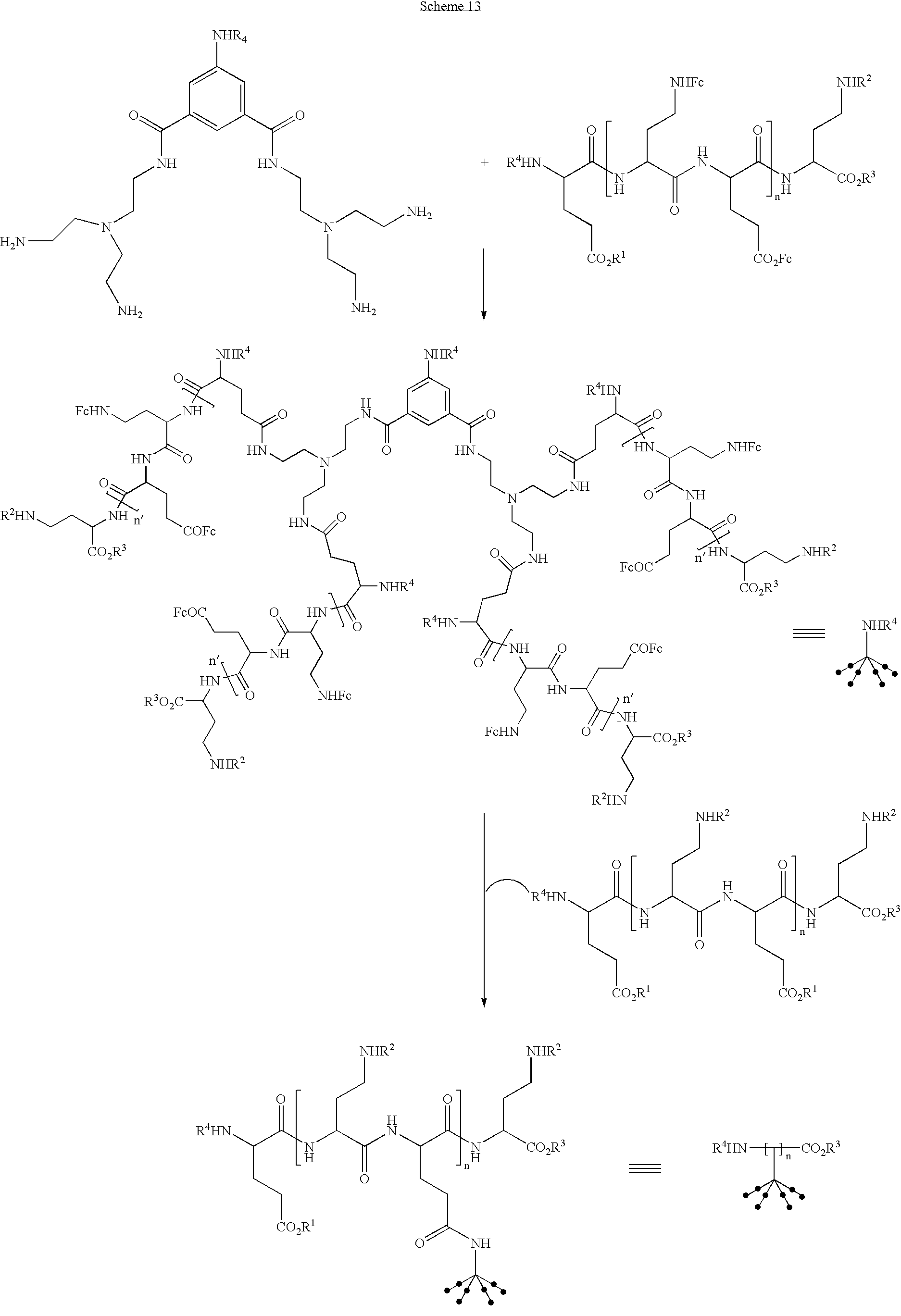

Similarly, it is within the scope of the invention to include all variations in which the singular scaffold has a serial or serial-parallel format from which indicator moieties or other formats append therefrom to avail other indicator motifs and topology.

### Covalent Arrangements

In certain embodiments, the targeted reporters of the present invention can be constructed without use of a coordinating metal center.

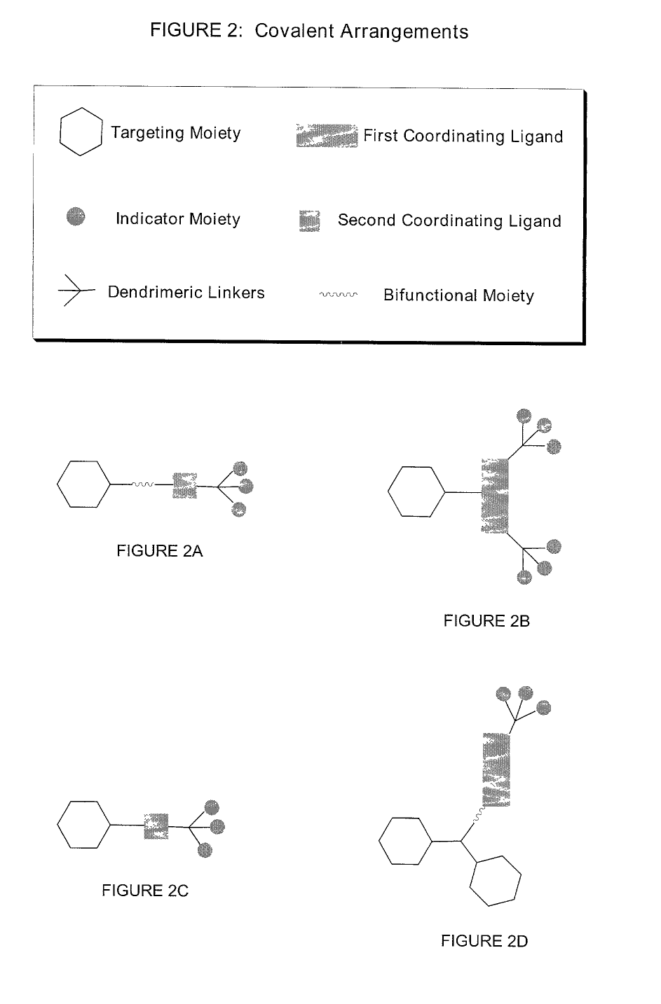

FIG. 2 schematizes several classes of covalent molecular topologies.

FIG. 2A schematically depicts a covalent arrangement in which the targeting moiety is bonded to a dendritic signal amplifier moiety through a bifunctional linker, which can usefully comprise a carbohydrate or polypeptide polymer. The bifunctional linker can serve to elongate the branch, conferring flexibility, and can function as a branch point for the attachment of additional dendritic signal amplifiers or direct attachment of indicator moieties.

FIG. 2B shows the direct attachment of the targeting moiety to a first coordinating ligand having two dendritic signal amplifier moieties bonded thereto, without the intermediation of a bifunctional linker. In this topology, the target moiety is in closer proximity to the indicator moieties; where the indicator moieties are redox active, electrons can in certain embodiments flow through the bonds, placing the indicator moieties in electrical communication with the targeting moiety. FIG. 2C is to similar effect.

For example, the targeting moiety can be bonded directly to the signal amplifier according to scheme (14):

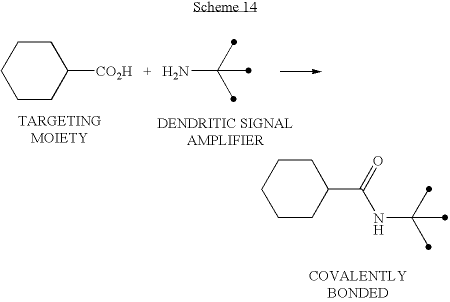

Although scheme (14) uses an amide bond to link the targeting moiety to the amplifier moiety, it would be understood that other bonding reactions can be used, with ester, urea, carbamate, thiocarbamate, thiourea, thioamide, double bonds and carbonate bonds proving particularly useful.

Alternatively, or in addition, multiple targeting moieties can be incorporated within a single targeted reporter of the present invention, as schematized in FIG. 2D. As an example of the latter, a combination of nucleic acid intercalator, such as acridine, and an oligonucleotide as targeting moieties on a single reporter could enhance the binding specificity or magnitude of the interaction, since acridines are known to stabilize the formation of duplexes.

Optionally, since polyguanine can amplify signals and enhance solubility, oligonucleotide hybridization and signal amplification can be combined into one targeting moiety by using an single oligonucleotide sequence having a polyguanine tail (Scheme 15):

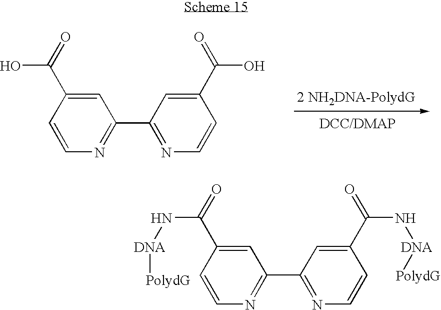

With this arrangement, the indicator moieties pendant from the termini of the dendrimer would not be needed, and the dendrimer, if present, can then solely function to enhance molecular solubility.

It is also possible to separate the targeting oligonucleotide moiety from the polyguanine.

In addition, the dendritic signal amplifier moiety, by itself, can be used as a label or marker, wherein the amplifier moiety is designed to bind covalently or non-covalently to a pool of targets or a selective group of targets within a pool of molecules. Once these targets are bound to the amplifier moiety, they are allowed to hybridize to targeting moieties free in solution or immobilized on an electroactive or transducing surface, such as an electrode, microarray, carbon tubes and carbon nanowires, forming duplexes or complementary pairs with the corresponding targeting moiety. Depending on the degree of complementarity, each pair will elicit different intensity and duration of signals in the presence of either an applied voltage or current.

In conjunction with the use of amplifier moieties as a label, a plurality of labels, wherein each label has a unique electro-optical signal or signature, can be introduced to react or recognize a specific set of complementary species having a particular functional group, sequence, moiety, folding or configuration, in a pool of targets. This can, in practice, provide a rapid and sensitive means for mining and identifying a specific set or a plurality of targeting moieties, such as drugs, peptides, metabolites and genes, from a pool of targets.

Compositions

In another aspect, the invention provides compositions of the targeted dendrimeric reporters of the present invention.

The targeted reporters of the present invention are typically soluble in aqueous solvents, especially those targeted dendrimeric reporters useful for electrochemical sensing of nucleic acid and other biologically-relevant recognition events. For such reporters, the composition of the present invention thus comprises, in its simplest formulation, the targeted reporter admixed with an aqueous solvent, such as water. Other protic and aprotic polar solvents, such as ethanol, N-methylpyrrolidone, and DMSO, can also readily be used.

The compositions can further comprise agents that facilitate targeting.

For targeting nucleic acids, such additional agents include, e.g., monovalent salts, such as NaCl and NaOAc, that serve to decrease hybridization stringency, divalent salts, such as magnesium, to facilitate hybridization, surface-active agents, such as detergents, and hybridization accelerants, such as cetyltrimethylammonium bromide (CTAB), U.S. Pat. Nos. 5,474,911, 5,015,569, and 5,747,254, to facilitate duplex formation, and preservatives, such as the cationic chelators EDTA and EGTA, or other preservatives known in the art, such as sodium azide.

The compositions of the present invention can include an admixture of a plurality of targeted reporters with different targeting and/or different indicator moieties, permitting multiplex analysis.

The compositions of the present invention can also include agents for catalytically regenerating the indicator moieties.

Electrochemical Sensing Using Targeted Dendrimeric Reporters

The targeted dendrimeric reporters of the present invention can be used in a wide variety of electrochemical sensing methods.

In general, the method comprises contacting a sample to be queried for the presence of a chosen analyte with a composition that comprises a dendrimeric reporter of the present invention, and then electrochemically detecting the indicator moieties of the dendrimeric reporter molecules that are targeted to the chosen analyte.

For example, to detect a nucleic acid target of specific sequence, a dendrimeric reporter of the present invention can usefully have a nucleic acid intercalator, such as dipyrido[3,2-a:2′,3′-c]phenazine, as targeting moiety, and ferrocene or polyguanine as an electroactive indicator moiety. The dendrimeric reporter is added to a nucleic acid sample, and the admixture contacted by an electrode that is covalently modified with a single-stranded nucleic acid probe complementary in sequence to the target nucleic desired to be detected.

If the sample contains the target nucleic acid, duplex formation at the electrode acts to concentrate the duplex-targeted dendrimeric reporter at the electrode, where the dendritically ramified ferrocenyl indicator moieties produce detectable current.

The targeted reporter can, alternatively, be added after contact of the sample. In another variant, a further redox active agent can be added in order catalytically to regenerate the indicator moieties, extending the duration of current flow.

Oligonucleotide-modified electrodes, and methods for their construction, are described, inter alia, in U.S. Pat. No. 5,312,527; Armistead et al., Anal. Chem. 72:3764-3770 (2000); Yang et al., Anal. Chem. 73(21):5316-22 (2001); and Yamashita et al., Nucl. Acids Symposium Series No. 42:185-186 (1999), and reviewed in Pividori et al., Biosensors & Bioelectronics 15:291-303 (2000), the disclosures of which are incorporated herein by reference in their entireties.

Electrical measurements can be made using a number of approaches well known in the art.

For example, measurements can be made by cyclic voltammetry, applying a triangle electrode potential wave and measuring current passing through the electrode. Data is typically presented by plotting current vs. potential, and the signal from the redox indicator moieties has a pair of reduction and oxidation waves at a charcteristic electropotential.

Alternatively, electrical activity can be measured using chronopotentiometry, in which a constant current is applied and the potential measured at the working electrode versus time. The signal from the specific redox indicator corresponds to a peak at a characteristic position.

In yet another alternative, chronoamperometry can be used. In chronoamperometry, a constant electrode potential is applied and current measured at the working electrode as a function of time. The signal from redox indicators is obtained by subtracting the data from a control measurement.

Another approach is to use AC voltammetry, in which an extra sinusoidal AC wave is applied on top of the triangle electrode potential wave; current passing through the electrode is measured after a lock-in amplifier. Data is typically presented as current vs. potential. The signal from the specific redox indicator moeities has a peak at a characteristic electropotential. Since each electron in the redox species concentrated at the electrode surface can be used many times (due to AC modulation), sensitivity can be significantly better than that with standard DC cyclic voltammetry.

As another example, dendrimeric reporters having nucleic acid intercalators as targeting moieties and electroactive indicator moieties, such as ferrocence, can be used in place of daunomycin to amplify signals in the methods of Kelley et al., Nucl. Acids Res. 27(24):4830-4837 (1999), Boon et al., Nature Biotechnol. 18:1096-1100 (2000), Boon et al., Nature Biotechnol. 20:282-286 (2002), the disclosures of which are incorporated herein by reference in their entireties.

If the targeting moiety instead comprises a single-stranded nucleic acid complementary in sequence to a desired target nucleic acid, and the reporter includes an electroactive moiety, such as ferrocene, as indicator, the targeted reporter of the present invention can be readily be used in the methods of Ihara et al., Chem. Commun. 1997, 1609-1610, the disclosure of which is incorporated herein by reference in its entirety. Briefly, in a sandwich assay, the target oligonucleotide binds simultaneously to its cognate probe at the electrode surface, and to the ferrocenyl oligonucleotide dendrimeric reporter; the targeting moiety of the reporter binds elsewhere on the target. This target-mediated dual recognition raises the electrode-local concentration of the electroactive ferrocene moiety, increasing the oxidation current.

The targeting moiety of the dendrimeric reporters of the present invention is not limited to those that are specific for nucleic acids; the targeted dendrimeric reporters of the present invention thus find use in electrochemical sensing of other classes of analytes, such as proteins.

In other variants, the targeted reporters of the present invention, rather than freely diffusing to the electrode, are instead immobilized thereto. In such case, it is the analyte that is concentrated at the electrode surface by the targeting moiety, and the concentrated analyte acts to alter the steady-state current generated by the indicator moieties.

Kits

In another aspect, the present invention provides kits to facilitate electrochemical detection, and optionally quantitation, of target.

Typically, the kits comprise one or more compositions, each comprising a distinct targeted dendrimeric amplifier of the present invention.

For nucleic acid detection, the kits can also comprise, packaged separately therefrom, one or more additional compositions that comprise agents that facilitate targeting, such as monovalent salts, such as NaCl and NaOAc, divalent salts, such as magnesium, surface-active agents, such as detergents, and hybridization accelerants, such as cetyltrimethylammonium bromide (CTAB), U.S. Pat. Nos. 5,474,911, 5,015,569, and 5,747,254.

Kits intended to facilitate detection of nucleic acids can also usefully contain oligonucleotides.

The oligonucleotide can, for example, have a region complementary in sequence to a probe immobilized on an electrode, and a region complementary in sequence to an oligonucleotide targeting moiety of the targeted dendrimeric reporter. The additional oligonucleotide serves, as in Ihara et al., Chem. Commun. 1997, 1609-1610, to secure the targeted dendrimeric reporter to the electrode-immobilized probe in a sandwich assay.

As another example, an oligonucleotide included in the kit can be identical in sequence to an electrode-immobilized probe, and thus be used in varying concentration to compete with the electrode-immobilized probe for binding of the targeted dendrimeric reporter to provide confirmation of specificity.

In yet another alternative, the oligonucleotide included in the kit can differ in sequence, as by a single nucleotide, from an electrode-immobilized probe, permitting analysis by competition of the specificity of target binding.

The kits of the present invention can also usefully include agents that permit catalytical regeneration of the indicator moieties of the targeted dendrimeric reporters.

Typically, the kits will also include information, typically in the form of a package insert, on the electropotential of the indicator moieties, and recommended conditions for monitoring their binding. In other embodiments, the kits of the present invention can include dendritic amplifiers in a form suitable for attachment to a targeting moiety to be provided by the user.

In other embodiments, the kits of the present invention can include the electrochemical transducer, or sensor. In such kits, the targeted dendrimeric reporters can be packaged separately from the electrode, or can be immobilized to the sensor, either non-covalently or covalently. The surface of the sensor can be adapted for the attachment of the targeted reporters.

### EXAMPLES

### Examples 1-2

Examples 1-2, as illustrated in schemes 16 and 17, respectively, describe various coordinative arrangements of first- and second-coordinating ligands surrounding a tetra-coordinating metal center such as platinum. For illustrative purposes, dipyrido[3,2-a:2′,3′-c]phenazine, a strong bidentate targeting moiety, is used.

### Example 1

This example shows the partial synthesis of targeted reporters having a tetracoordinating platinum metal center coordinatively bonded to a bidentate intercalator as targeting moiety and a bidentate first-coordinating ligand. For complete synthesis, R is a dendritic amplifier or directly attached indicator moiety.

Compound 20 is synthesized according to the method of Hiort et al., J. Am. Chem. Soc. 115(9):3448-3454 (1993). Stirring a solution of platinum (II) chloride, dipyrido[3,2-a:2′,3′-c]phenazine and a disubstituted bipyridine gives compound 20 as a dichloride complex.

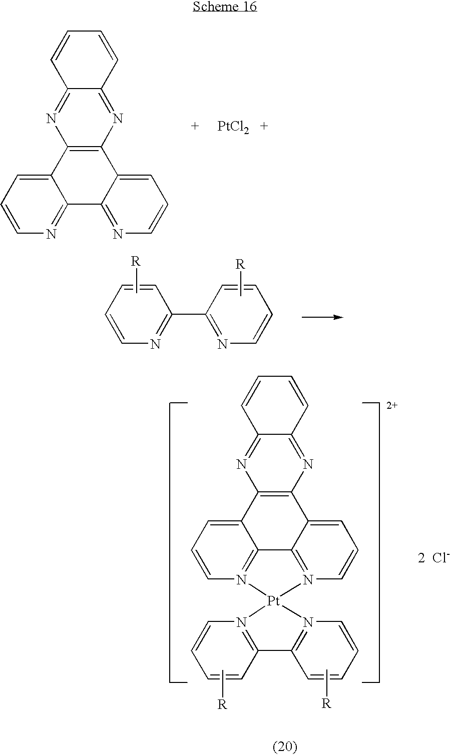

### Example 2

This example shows the partial synthesis of targeted reporters having a tetracoordinating platinum metal center coordinatively bonded to a bidentate intercalator as targeting moiety and two monodentate second-coordinating ligands. For complete synthesis, R is a dendritic amplifier or directly attached indicator moiety.

Using the method of Hiort et al., J. Am. Chem. Soc. 115(9):3448-3454 (1993), compound 21 is synthesized by adding approximately two equivalents of a second-coordinating amine ligand to a solution of platinum (II) chloride and dipyrido[3,2-a:2′,3′-c]phenazine.

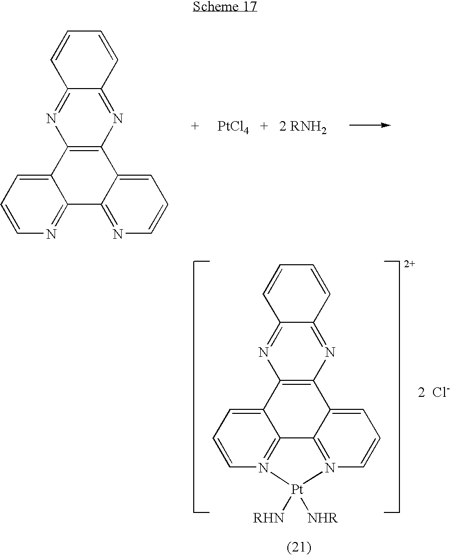

### Examples 3-6

Examples 3-6, illustrated respectively in schemes 18-21, describe various coordinative arrangements of first- and second-coordinating ligands surrounding a hexa-coordinating metal center such as ruthenium. For illustrative purposes, dipyrido[3,2-a:2′,3′-c]phenazine, a strong bidentate targeting moiety, is used.

### Example 3

This example shows the partial synthesis of targeted dendrimeric reporters having a hexacoordinating ruthenium metal center coordinatively bonded to a bidentate intercalator as targeting moiety and two bidentate first-coordinating ligands. For complete synthesis, R is a dendritic signal amplifier or indicator moiety.

Mixing together ruthenium (II) chloride, dipyrido[3,2-a:2′,3′-c]phenazine and approximately two equivalents of a first-coordinating bipyridine ligand, according the method of Hiort et al., J. Am. Chem. Soc. 115(9):3448-3454 (1993), gives compound 22.

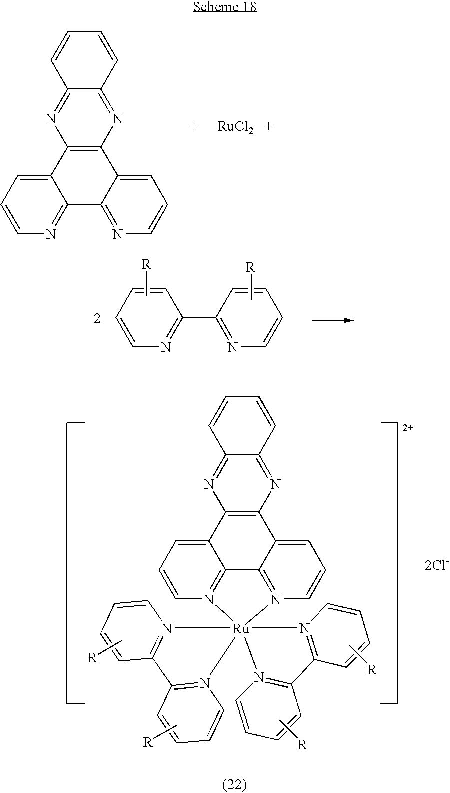

### Example 4

This example shows the partial synthesis of targeted dendrimeric reporters having a hexacoordinating ruthenium metal center coordinatively bonded to a bidentate intercalator and sequentially complexed with one bidentate first-coordinating ligand followed by complexation with two monodentate second-coordinating ligands; for complete synthesis, R and R′ are each dendritic signal amplifiers or indicator moieties such as, but not limited to, ferrocene or oligonucleotide-polydG.

Mixing together ruthenium (II) chloride, dipyrido[3,2-a:2′,3′-c]phenazine and bipyridine, according the method of Hiort et al., J. Am. Chem. Soc. 115(9):3448-3454 (1993), yields a dichloro-intermediate as shown in scheme 19, which upon treatment with approximately two equivalents of an oligonucleotide-polydG, a second-coordinating ligand, gives compound 23.

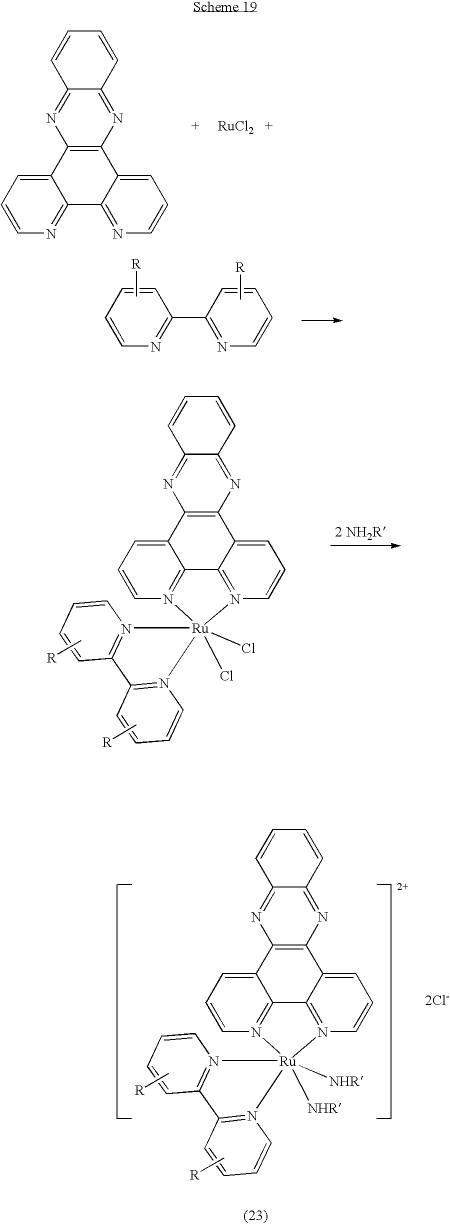

### Example 5

This specific example, as shown in scheme 20, demonstrates the utility of the invention wherein oligonucleotides-polydG having a free amine group can act as a second-coordinating ligand as well as a sensing moiety.

Mixing together ruthenium (II) chloride, dipyrido[3,2-a:2′,3′-c]phenazine and bipyridine, according the method of Hiort et al., J. Am. Chem. Soc. 115(9):3448-3454 (1993), yields a dichloro-intermediate which, upon treatment with approximately two equivalents of an oligonucleotide-polydG, a second-coordinating ligand, yields compound 24 (Scheme 20).

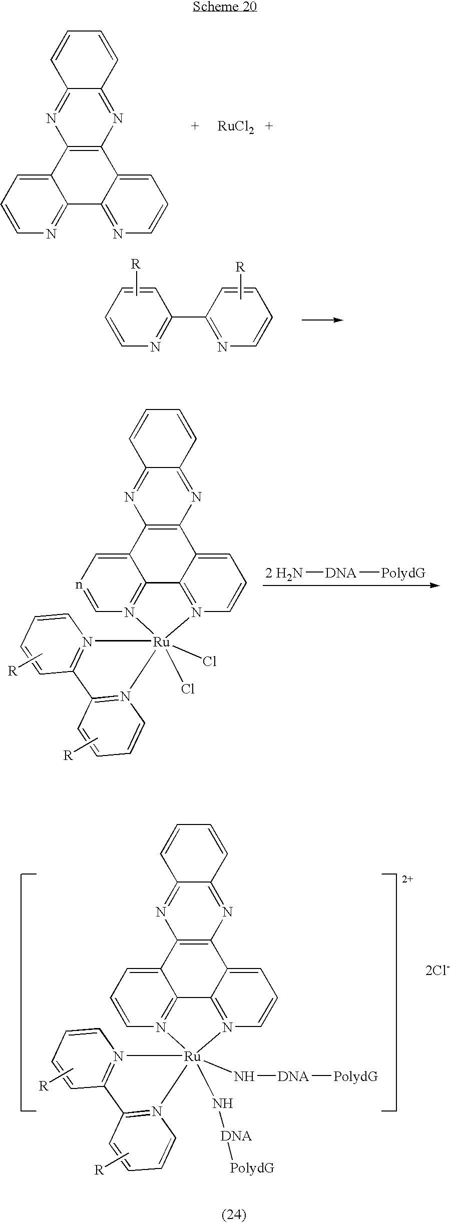

### Example 6

This example shows the partial synthesis of signal amplifying targeted reporters having a hexacoordinating ruthenium metal center coordinatively bonded to a bidentate intercalator and four monodentate second-coordinating ligands; for complete synthesis, R is a dendritic signal amplifier or indicator moiety.

Mixing together ruthenium (II) chloride, dipyrido[3,2-a:2 ′,3′-c]phenazine and approximately four equivalents of a second-coordinating amine ligand, according the method of Hiort et al., J. Am. Chem. Soc. 115(9):3448-3454 (1993), affords compound 25.

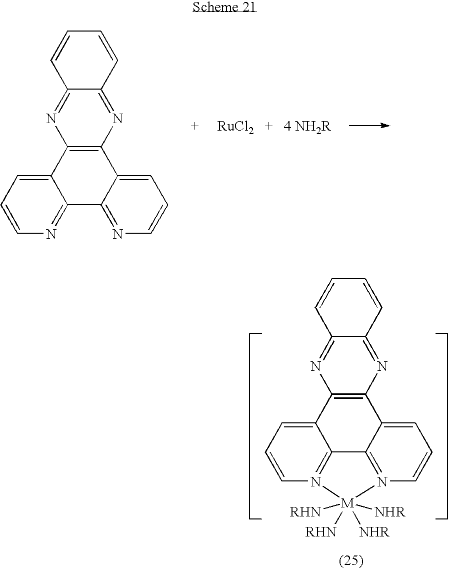

### Example 7

Example 7 is illustrated in scheme 22. Mesylation of 4,4′-dihydroxymethyl-2,2′-bipyridine renders the bipyridine susceptible to nucleophilic attack by alcohols and amines.

Example 7. This example demonstrates a useful mode of covalent bond formation in the synthesis of targeted dendrimeric reporters, in which 4,4-dihydroxymethyl-2,2′-bipyridine, a bidentate coordinating ligand, acting as an electrophile, reacts upon mesylation with an alcoholic nucleophile.

Using the method of Ghosh et al., J. Am. Chem. Soc. 102(17):5543-5549 (1980), the product generated from the mesylation of 4,4′-dihydroxymethyl-2,2′-bipyridine with mesyl chloride, in the presence of triethylamine, reacts readily with either an amine or an alcoholic nucleophile to give the corresponding N-alkylated compound (26) or the O-alkylated compound (27) respectively.

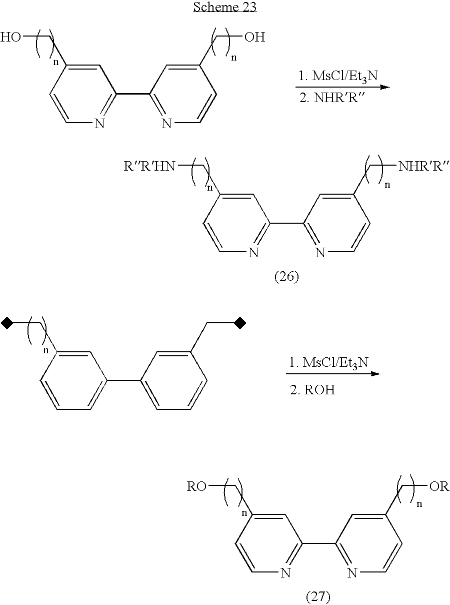

### Example 8

Example 8. 2,2′-bipyridyl-4,4′-dicarboxylic acid is a useful intermediate in the synthesis of targeted dendrimeric reporters, and their dendritic signal amplifiers.

This example demonstrates a convenient method of converting 4,4′-dimethyl-2,2′-bipyridine into bipyridyl-4,4′-dicarboxylic acid according to the method of Oki et al., Synth. Commun. 25(24):4093-4097 (1995). This example also describes an efficient and mild means of coupling nucleophiles, such as amines and alcohols, to first-coordinating bipyridine ligands, having carboxylic acid moieties, using dicyclohexylcarbodiimide.

The addition of either an amine or an alcohol nucleophile to a mixture of dicyclohexylcarbodiimide (DCC), dimethylaminopyridine and 2,2′-bipyridyl-4,4′-dicarboxylic acid, generated from the oxidation of 4,4′-dimethyl-2,2′-bipyridine, gives the corresponding amide or ester product, respectively, as shown in Scheme 24.

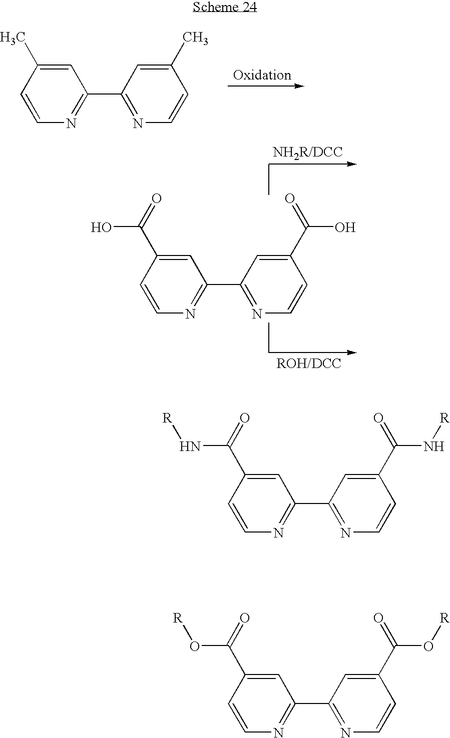

### Example 9

Example 9. Example 9 is illustrated in Scheme 25. By virtue of the dihydroxyl groups, 4,4′-dihydroxymethyl-2,2′-dipyridine is inherently nucleophilic, and therefore readily reacts with electrophilic molecules such as isocyanates to form a covalent bond.

Combining a solution of 4,4′-dihydroxymethyl-2,2′-dipyridine with an excess amount of an isocyanate, according to the method of Ghosh et al., J. Am. Chem. Soc. 102(17):5543-5549 (1980), gives a carbamate product as shown in scheme 25.

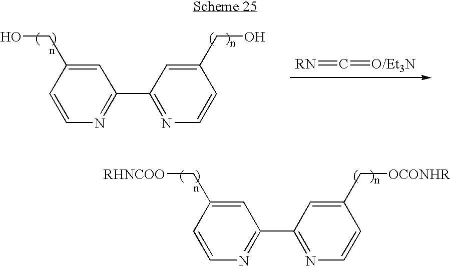

### Example 10

Example 10. Example 10 is illustrated in Scheme 26. This example demonstrates that by acetylating 4,4′dihydroxymethyl-2,2′dipyridine with acetyl chloride, the molecule becomes susceptible to chemical bond formation with a nucleophile of choice through a displacement reaction.

Acetylation of dihydroxymethyl-2,2′-dipyridine with acetyl chloride in the presence of triethylamine, according to the method Smith et al., Tet. Lett. 41(16):2787-2789 (2000), affords a diacetylated intermediate as shown in Scheme 26. Subsequent reaction with a nucleophile, such as an amine or alcohol, gives the corresponding N-alkylated amine or O-alkylated ether product.

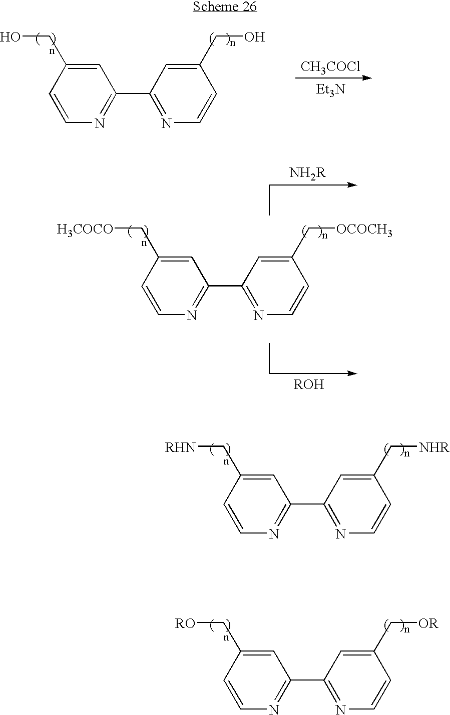

All patents, patent publications, and other published references mentioned herein are hereby incorporated by reference in their entireties as if each had been individually and specifically incorporated by reference herein.

Although illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. A targeted dendrimeric reporter, comprising:
a targeting moiety; and
at least one dendritic signal amplifier,
wherein each of said at least one dendritic signal amplifiers is linked to said targeting moiety.

2. The dendrimeric reporter of claim 1, further comprising at least one linking ligand, wherein each of said at least one dendritic amplifiers is linked to said targeting moiety by at least one of said at least one ligands.

3. The dendrimeric reporter of claim 2, further comprising a metal center, wherein said metal center commonly coordinates said targeting moiety and each of said linking ligands.

4. The dendrimeric reporter of claim 3, wherein said metal center is a hexacoordinating metal center.

5. The dendrimeric reporter of claim 4, having either of the formulae: 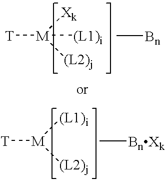
wherein
T is a targeting moiety;
M is a metal center;
X is a counterion;
L1 is a first coordinating ligand, said first ligand having two coordination sites;
L2 is a second coordinating ligand, said second ligand having one coordination site;
B is a dendritic amplifier;
i is an integer from 0-2;
j is an integer from 0-5;
at least one of i or j is greater than or equal to 1;
k is an integer from 0-4;
n is an integer greater than 1; and
said metal center participates in a total of six coordinate bonds.

6. The dendrimeric reporter of claim 4 or claim 5, wherein M is Ru.

7. The dendrimeric reporter of claim 3, wherein said metal center is a tetracoordinating metal center.

8. The dendrimeric reporter of claim 7, having either of the formulae: 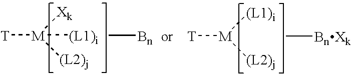
wherein
T is a targeting moiety;
M is a metal center;
X is a counter ion;
L1 is a first coordinating ligand, said first ligand having two coordination sites;
L2 is a second coordinating ligand, said second ligand having one coordination site;
B is a dendritic amplifier;
i is an integer from 0-1;
j is an integer from 0-3;
at least one of i or j is greater than or equal to 1;
k is an integer from 0-2;
n is an integer greater than 1; and
said metal center participates in a total of four coordinate bonds.

9. The dendrimeric reporter of either of claims 7 or 8, wherein M is Pt.

10. The dendrimeric reporter of claim 5 or claim 8, wherein L1 is selected from the group consisting of 1,2-diaminobenzenes, 1,2-diaminoethanes, bipyridines, phenathrolines, biquinoline, and pyrazines; and L2 is selected from the group consisting of pyridines, quinolines, piperidines, morpholines, pyrrolidines, pyrroles, pyrazines, thiophenes, piperazines, tetrahydrofurans, furans, phenols, and amines.

11. The dendrimeric reporter of any one of claims 1, 5, or 8, wherein said targeting moiety is a nucleic acid intercalator.

12. The dendrimeric reporter of claim 11, wherein said nucleic acid intercalator is selected from the group consisting of: daunomycin, phenothiazinium dye, viologen, phenanthridines, phenothiazines, phenazines, acridines, anthraquinones, napththalenyls, and oxazolopyridocarbazoles.

13. The dendrimeric reporter of claim 12, wherein said intercalator is dipyrido[3,2-a:2′,3′-c]phenazine.

14. The dendrimeric reporter of any one of claims 1, 5, or 8, wherein said targeting moiety is an oligonucleotide.

15. The dendrimeric reporter of any one of claims 1, 5, or 8 wherein said targeting moiety is an antibody, or antigen-binding fragment thereof.

16. The dendrimeric reporter of any one of claims 1, 5, or 8, wherein said dendritic amplifier comprises a core, a plurality of dendritic branches ramifying from said core, and a plurality of indicator moieties, each of said indicator moieties bonded to a dendritic branch, wherein the core of said dendritic amplifier is selected from the group consisting of sugars and benzylamines.

17. The dendrimeric reporter of claim 16, wherein the core of said dendritic amplifier is a hexose sugar.

18. The dendrimeric reporter of claim 17, wherein the bifunctional moiety of said dendritic amplifier branches is a peptide.

19. The dendrimeric reporter of claim 15, wherein said indicator moieties are redox active.

20. The dendrimeric reporter of claim 19, wherein said indicator moiety includes ferrocene or polyguanine.

21. The dendrimeric reporter of claim 20, wherein said indicator moiety includes ferrocene.

22. A targeted dendrimeric reporter, comprising:
targeting means; and
dendritic signaling means bonded thereto.

23. A method of detecting the presence of a chosen analyte in a sample, the method comprising:
contacting a sample to be queried for the presence of a chosen analyte with a composition that comprises a targeted dendrimeric reporter, said dendrimeric reporter comprising a targeting moiety and at least one dendritic signal amplifier; and then
electrochemically detecting the indicator moieties of the dendrimeric reporter molecules that are targeted to the chosen analyte.

24. The method of claim 23, wherein said analyte is a nucleic acid.

25. The method of claim 24, wherein said targeting moiety is specific for double-stranded nucleic acids.

26. The method of claim 25, wherein said targeting moiety is a nucleic acid intercalator.

27. The method of claim 26, wherein said intercalator is a phenazine.

28. The method of claim 26 or claim 27, wherein said step of electrochemically detecting comprises:
contacting both said sample and said intercalating dendrimeric reporter to an electrode, said electrode covalently modified with an oligonucleotide sufficiently complementary in sequence to hybridize to said nucleic acid analyte; and
detecting current flow through said electrode.

29. The method of claim 28, wherein said dendritic amplifier comprises a dendritic core, a plurality of dendritic branches ramifying from said core, and a plurality of indicator moieties, each of said indicator moieties bonded to a dendritic branch, wherein at least a plurality of said indicator moieties are ferrocenes.

30. In a method of electrochemical detection of an analyte, the improvement comprising:
contacting the electrochemical sensor, in the presence of analyte, with a targeted dendrimeric reporter, said dendrimeric reporter comprising a targeting moiety and at least one dendritic signal amplifier.

31. A method of making a targeted dendrimeric reporter, the method comprising:
linking a targeting moiety to a dendritic signal amplifier through a coordinating metal center.

32. A kit for electrochemical detection of an analyte, comprising:
at least one composition comprising a targeted dendrimeric reporter; and
indicia that identify at least one parameter required to detect the indicator moieties of said dendrimeric reporter.

33. The kit of claim 32, wherein a plurality of indicator moieties of said targeted dendrimeric reporter are redox active, and said at least one parameter identifies an electrochemical potential of said indicators.

34. The kit of claim 33, wherein the targeting moiety of said targeted dendrimeric reporter has binding specificity for nucleic acid.

35. The kit of claim 34, further comprising:
at least one composition that faciliates nucleic acid hybridization, said composition including an agent selected from the group consisting of monovalent salts, NaCl, NaOAc, divalent salts, magnesium, surface-active agents, detergents, hybridization accelerants, and cetyltrimethylammonium bromide (CTAB).

36. The kit of claim 34, further comprising:
a composition comprising an oligonucleotide.

37. The kit of claim 33, further comprising:
a composition comprising a redox active agent capable of catalytically regenerating the electrochemically active indicator moiety of said targeted dendrimeric reporters.

38. The kit of claim 33, further comprising an electrochemical sensor.

39. The kit of claim 38, wherein said electrochemical sensor includes electrodes consisting of nanotubes or nanowires.
